(19) United States
(12) Patent Application Publication
(10) Pub. No.: US 2024/0246879 A1
(43) Pub. Date: Jul. 25, 2024

(54) PLASTIC DIGESTION

(71) Applicant: NBTECH AB, Järfälla (SE)

(72) Inventors: Nathalie BEREZINA, Järfälla (SE); Maxim CHAPOVALOV, Järfälla (SE)

(21) Appl. No.: 18/566,579

(22) PCT Filed: Jul. 1, 2022
(86) PCT No.: PCT/EP2022/068326

§ 371 Date: Dec. 1, 2023

(30) Foreign Application Priority Data
Jul. 2, 2021 (EP) 21183517.8
Jul. 2, 2021 (EP) 21183520.2
Nov. 25, 2021 (EP) 21210560.5

(51) Int. Cl. C05F 17/05; B09B 3/60; B09B 101/75; B09B 101/77; C05F 9/00

(52) U.S. Cl. CPC C05F 17/05; B09B 3/60; C05F 9/00; B09B 2101/75; B09B 2101/77

(57) ABSTRACT

The present disclosure relates to degradation of polymer materials, such as plastic materials. More specifically, the present disclosure relates to a method for digestion of one or more plastic-comprising material by the use of at least one larva of the species selected from the group consisting of Galleria mellonella, Tenebrio molitor and Alphitobius diaperinus. The product obtained by the method is also within the inventive concept. The disclosure furthermore relates to use of said at least one larva for digestion of a plastic-comprising material.

### TECHNICAL FIELD

The inventive concept described herein generally relates to degradation of polymer materials, such as plastic materials. More specifically, the present disclosure relates to a method for digestion of one or more plastic-comprising material by the use of at least one larva of the species selected from the group consisting of Galleria mellonella, Tenebrio molitor and Alphitobius diaperinus. The product obtained by the method is also within the inventive concept. The disclosure furthermore relates to use of said at least one larva for digestion of a plastic-comprising material.

### BACKGROUND

Plastics are synthetic polymers derived from fossil oil or renewable materials and largely resistant to biodegradation. Because of this resistance, plastics have a heavy environmental impact. Plastic pollution is a serious problem, and worsening at rapid rate. Estimates state that by 2050 the world's oceans will contain more plastic than fish. Solutions to the problem of the plastic degradation and/or recycling are thus urgently needed. However, hundreds of different plastics exist and different plastics require different treatment for degradation and/or recycling. Plastic products often contain more than one part comprising different plastic polymers, making it difficult to separate the different plastics for degradation and/or recycling. Also, many products comprise mixes of plastic polymers and/or copolymers. Additionally, the mixed way in which plastics are collected makes recycling very challenging, as the various types are difficult to separate. Thus, a large quantality of plastic materials is neither degraded or recycled and thus ends up polluting the environment.

Galleria mellonella, with the common name greater wax moth, is a member of the family Pyralidae, which is a pest of the honeybee. It feeds on beeswax in the wild, i.e., mainly on esters formed by esterification of fatty acids with fatty alcohols. While being considered as a pest in the wild, it has been more appreciated in laboratory environments. G. mellonella is well-accepted as an insect model for studies relating to pathogen-host interactions and studies relating to antimicrobial compounds. The insect provides several advantages such low cost of maintenance, fast life cycle, the possibility of using a large number of larvae and an innate immune system which evolutionarily conserved relative to mammals.

WO 2014/067081 discloses insects capable of degrading petroleum-based plastics. The insects can be snout moths and the petroleum-based plastic can be amongst other polypropylene, polyurethane, polyamide or the polyacrylate polymethylmethacrylate.

In 2017, one group of researchers (Bombelli et al.) discovered that G. mellonella was able to consume polyethylene (PE) by the observance of holes in a plastic bag containing larvae. In 2020, another group of scientists (Lou et al.) have shown that G. mellonella is able to digest polystyrene (PS). The article teaches that larvae survival decreased when G. mellonella was feed polystyrene or polyethylene, compared to when G. mellonella was feed on beeswax or starved. Supplementing the plastic feed of G. mellonella with beeswax or bran, the survival rate of the larvae increased compared to when the feed was only polystyrene or polyethylene. As such, G. mellonella exhibits a preference away from feeding on plastics.

Although effort has been made in the field of plastic degradation, there is an enormous need for improved methods for degradation of plastic. In particular, method which allow for degradation of different types of as well as mixed plastic materials are needed. Furthermore, improved degradation methods would desirably also provide useful end products.

### DISCLOSURE OF THE INVENTION

It is an object of the present invention to at least partly reduce or overcome challenges in the prior art, and provide means for degrading one or more plastic-comprising material. This object is achieved as disclosed herein by the inventive use of at least one larva of the species G. mellonella for degradation of plastic-comprising material.

It is another object of the present invention to provide means for degrading one or more plastic-comprising material.

These and other objects, which will be apparent to a skilled person from the present disclosure, are achieved by the different aspects of the disclosure as defined in the appended claims and as generally disclosed herein.

In a first aspect of the disclosure, there is provided a method for degrading one or more plastic-comprising material. The method comprises contacting at least one larva of the species G. mellonella with at least one or more plastic-comprising materials under conditions permissive for said at least one larva to digest said material. The material comprises at least one plastic polymer selected from the group consisting of polyethylene terephthalate, polypropylene, polypropylene terephthalate, polyether, polyurethane, polyester, polyacrylate, polyglycol, polyvinyl chloride, polyvinyl acetate, polycarbonate, polyvinylidene chloride, synthetic polyamide and any mixes or copolymers thereof. In one embodiment, the material comprising at least one plastic polymer is selected from the group consisting of polyethylene terephthalate, polypropylene, polypropylene terephthalate, polyether, polyurethane, polyacrylate, polyglycol, polyvinyl chloride, polyvinyl acetate, polycarbonate, polyvinylidene chloride, synthetic polyamide and any mixes or copolymers thereof. In one embodiment, the material comprising at least one plastic polymer is selected from the group consisting of or selected from the group consisting of polyethylene terephthalate, polypropylene, polypropylene terephthalate, polyether, polyurethane, polyester, polyacrylate, polyglycol, polyvinyl chloride, polycarbonate, polyvinylidene chloride, synthetic polyamide and any mixes or copolymers thereof. In one embodiment, the material comprising at least one plastic polymer is selected from the group consisting of polyethylene terephthalate, polypropylene terephthalate, polyether, polyurethane, polyester, polyacrylate, polyglycol, polyvinyl chloride, polycarbonate, polyvinylidene chloride, synthetic polyamide and any mixes or copolymers thereof. In one embodiment, the material comprising at least one plastic polymer is selected from the group consisting of polyethylene terephthalate, polypropylene terephthalate, polyether, polyurethane, polyacrylate, polyglycol, polyvinyl chloride, polycarbonate, polyvinylidene chloride, synthetic polyamide and any mixes or copolymers thereof. In one particular embodiment, the copolymer of polyvinyl acetate is poly(ethylene-vinyl acetate). The present inventors also consider that larvae of the species Tenebrio molitor and/or Alphitobius diaperinus may be useful in the context of digestion of plastic-comprising materials. In other words, there is provided a method for degrading one or more plastic-comprising material, the method comprising

contacting at least one larva of the species selected from the group consisting of G. mellonella, Tenebrio molitor and Alphitobius diaperinus with at least one or more plastic—comprising materials under conditions permissive for said at least one larva to digest said material, wherein said material comprises at least one plastic polymer selected from the group consisting of polyethylene terephthalate, polypropylene, polypropylene terephthalate, polyether, polyurethane, polyester, polyacrylate, polyglycol, polyvinyl chloride, polyvinyl acetate, polycarbonate, polyvinylidene chloride, synthetic polyamide and any mixes or copolymers thereof. In one embodiment, the material comprising at least one plastic polymer is selected from the group consisting of polyethylene terephthalate, polypropylene, polypropylene terephthalate, polyether, polyurethane, polyacrylate, polyglycol, polyvinyl chloride, polyvinyl acetate, polycarbonate, polyvinylidene chloride, synthetic polyamide and any mixes or copolymers thereof. In one embodiment, the material comprising at least one plastic polymer is selected from the group consisting of or selected from the group consisting of polyethylene terephthalate, polypropylene, polypropylene terephthalate, polyether, polyurethane, polyester, polyacrylate, polyglycol, polyvinyl chloride, polycarbonate, polyvinylidene chloride, synthetic polyamide and any mixes or copolymers thereof. In one embodiment, the material comprising at least one plastic polymer is selected from the group consisting of polyethylene terephthalate, polypropylene terephthalate, polyether, polyurethane, polyester, polyacrylate, polyglycol, polyvinyl chloride, polycarbonate, polyvinylidene chloride, synthetic polyamide and any mixes or copolymers thereof. In one embodiment, the material comprising at least one plastic polymer is selected from the group consisting of polyethylene terephthalate, polypropylene terephthalate, polyether, polyurethane, polyacrylate, polyglycol, polyvinyl chloride, polycarbonate, polyvinylidene chloride, synthetic polyamide and any mixes or copolymers thereof.

Commonly, a plastic material may comprise more than one polymer. Commonly, a plastic material comprises mixes of polymers, or plastic copolymers. This is a problem for the waste industry because different plastics require different conditions and methods of treatment for degradation and/or recycling. As plastic products often contain many different plastic polymers (in the same or in different parts of the product) it is very challenging to sufficiently separate different plastics for efficient degradation and/or recycling. Additionally, the mixed way in which plastics are collected makes the recycling process very challenging due to mixed materials. This may lead to problems in degradation or recycling of the materials. Advantageously, the present disclosure provides a method for degradation of a range of plastic-comprising materials. Many plastic materials consist of more than one type plastic polymer, either as a mixture, composite material or copolymer. Thus, it is considered advantageous that such plastic material may be fully digested.

In one embodiment, the plastic-comprising material consists of at least one plastic polymer selected from the group consisting of polyethylene terephthalate, polypropylene, polyurethane, polyester, polyacrylate, polyglycol, polyvinyl chloride, polyvinyl acetate, polycarbonate, polyvinylidene chloride, synthetic polyamide, polypropylene terephthalate, polyether and any mixes or copolymers thereof; or is selected from the group consisting of polyethylene terephthalate, polypropylene, polyurethane, polyester, polyacrylate, polyglycol, polyvinyl chloride, polycarbonate, polyvinylidene chloride, synthetic polyamide, polypropylene terephthalate, polyether and any mixes or copolymers thereof; or is selected from the group consisting of polyethylene terephthalate, polyurethane, polyester, polyacrylate, polyglycol, polyvinyl chloride, polycarbonate, polyvinylidene chloride, synthetic polyamide, polypropylene terephthalate, polyether and any mixes or copolymers thereof; or is selected from the group consisting of polyethylene terephthalate, polyurethane, polyacrylate, polyglycol, polyvinyl chloride, polycarbonate, polyvinylidene chloride, synthetic polyamide, polypropylene terephthalate, polyether and any mixes or copolymers thereof.

Polyurethane is a group of polymers composed of urethane (carbamate) units, and comprises C—H, C—C, C—O and C—N chemical bonds. As shown in the Example section of the present application, G. mellonella is able to digest this polymer, and thus is able to break carbamate bonds. It is envisioned that G. mellonella is able to at least partially digest other polymers which comprise said chemical bond. Similarly, T. molitor and A. diaperinus is expected to be able to digest said polymers.

Polyamide is a group of polymers composed of amide units which specifically comprises amide bonds. While natural polyamides, such as proteins and other polypeptides, are polymers of amino acid units, synthetic polyamides are made up of the same repeating unit, which unit is not necessarily an amino acid. As such, this type of synthetic polymers may be more resistant to degradation. For example, nylons (such as PA 6; PA 6,6; PA 10 and PA 12), comprising repeating alkyl units, provides intermolecular hydrogen bonds between the oxygen of the carbonyl of one polymer or one chain of a polymer, to the hydrogen situated on nitrogen, on another polymer or another chain on the same polymer. This reduces availability of the material for degradation, since there are no functional groups that are easily accessed for degradation. This is apparent for the person of skill in the art, who knows that when one is unlucky to accommodate pests in one's wardrobe (in the form of for example the species Attagenus pellio) wool is often digested, but nylon or another polyamide intact. This is not only unfortunate for one's wardrobe, but may also be a problem when recycling an item (such as a garment) made of a copolymer comprising a natural polyamide (such as wool) in combination with a synthetic polymer (such as for example a polyether-polyurethane copolymer, elastane). According to the present disclosure, at least one larva of the species selected from the group consisting of Galleria mellonella, Tenebrio molitor and Alphitobius diaperinus is able to digest a copolymer comprising a natural polyamide and a synthetic polymer such as a polyether-polyurethane copolymer, elastane.

Moreover, it is shown in the appended examples that synthetic polyamides and mixes of synthetic polyamides together with a copolymer (as exemplified herein with a polyether-polyurethane copolymer, elastane), are digested by G. mellonella. Similarly, T. molitor and A. diaperinus is expected to be able to digest said polymers. Thus, according to the present disclosure, at least one larva of the species selected from the group consisting of Galleria mellonella, Tenebrio molitor and Alphitobius diaperinus is able to digest a copolymer comprising a synthetic polyamide. In one embodiment, said synthetic polyamide is an aliphatic polyamide. In one embodiment, said aliphatic polyamide is selected from the group consisting of PA66, PA6, PA46, PA610, PA612, PA11 and PA12. In one embodiment, said aliphatic polyamide is a copolymer.

In the appended examples, the polyamide is a non-aromatic polyamide. It is plausible, both in relation to the ability to degrade non-aromatic polyamides, as well as based on the ability of G. mellonella to degrade other aromatic-comprising polymers (such as polystyrene), that this ability is extended to other polyamides, such as semi-aromatic polyamides and aromatic polyamides. Similarly, T. molitor and A. diaperinus is expected to be able to digest said polymers. Examples of semi-aromatic polyamides that may be digested in accordance with the present disclosure are poly(hexamethylene teraphthalamide) (PA 6T) and poly(hexamethylene isophthalamide) (6I). Examples of aromatic polyamides that may be digested in accordance with the present disclosure are Kevlar (a para-armid) and Nomex (a meta-aramid).

In one embodiment, said polyamide is an aromatic polyamide. In one embodiment, said aromatic polyamide is selected from the group consisting of poly(m-phenylene isophthalamide), poly(p-phenylene terephthalamide), and copoly(p-phenylene/3,4′-diphenyl ether terephthalamide).

In one embodiment, said polyamide is a semi-aromatic polyamide. In one embodiment, said semi-aromatic polyamide is selected from the group consisting of poly(hexamethylene teraphthalamide) and poly(hexamethylene isophthalamide).

Polyester is a group of polymers composed of ester units and comprises C—H, C—C and C—O chemical bonds. As shown in the Example section of the present application, G. mellonella is able to digest this polymer, and thus is able to break ester bonds. It is envisioned that G. mellonella is able to at least partially digest other types of polyether and any polymers which comprise said chemical bond. Similarly, T. molitor and A. diaperinus is expected to be able to digest said polymers.

As shown in the appended examples, G. mellonella has the ability to degrade copolymer materials, for example poly(ethylene-vinyl acetate) (also referred to as EVA) and poly(acrylonitrile butandiene). Similarly, T. molitor and A. diaperinus is expected to be able to digest such copolymers.

Polyacrylate comprises C—H, C—C and C—O chemical bonds. As shown in the Example section of the present application, G. mellonella is able to digest this polymer, and thus is able to break C—H, C—C as well as C—O chemical bonds. It is envisioned that G. mellonella is able to at least partially digest other polymers which comprise said chemical bond. Similarly, T. molitor and A. diaperinus is expected to be able to digest said polymers.

Cotton is made up of cellulose, which is a polyether of β(1→4) linked D-glucose units. As shown in the Example section of the present application, G. mellonella is able to digest this polymer, and thus is able to break β(1→4) linked sugar bonds. It is envisioned that G. mellonella is able to at least partially digest other polymers which comprise said chemical bond. Similarly, T. molitor and A. diaperinus is expected to be able to digest said polymers.

As described in the examples and shown in the figures, digestion of one or more plastic-comprising material leads to larval mass evolution. As used herein, the term “larval mass evolution” refers to evolution of larval mass, which most often is an increase, but can also be a decrease or a maintenance of a specific mass. As shown in the Example section of the present disclosure, the larvae of G. mellonella which are fed a plastic polymer based feed increase more in mass than the larvae fed a wax-based feed, which is the natural feed of these larvae. This is shown in FIG. 2. Surprisingly, the mass increase of larvae that are fed exclusively on plastic (e.i. said material consist of plastic(s)) is achieved without supplementing said plastic with any supplementary feed (also referred to herein as additional feed). An supplementary feed is intended to mean a suitable feed for said larvae, such as a natural feed for said larvae. Thus, advantageously, the degradation of plastic materials is achieved by said larvae without any processing of the material before it is subjected to the degradation.

As said larvae are able to digest the herein disclosed plastic polymers, polymer mixes and copolymers, even without supplementing said plastic comprising material with any supplementary feed, the present invention provides advantages for plastic material degradation and recycling. Such advantage is for example that there is no need for the separation of different kinds of plastic materials when polymer mixes and/or copolymers. Another advantage is for example that there is no need to mix plastic materials subjected to degradation with a natural feed for said larvae, in order to achieve plastic digestion by said larvae. Furthermore, there is no need to mix plastic materials subjected to degradation with a supplementary feed (which may be a natural feed for the species) for obtaining increased larval mass.

The skilled person appreciates the advantages of digesting unwanted plastic polymers into the biological material of larvae. In one embodiment, biomass is obtained from said digestion. The biomass may comprise pupae, and/or larvae of the species G. mellonella, and/or feces of the larvae of the species G. mellonella. The biomass may comprise pupae, and/or larvae of the species T. molitor, and/or feces of the larvae of the species T. molitor. The biomass may comprise pupae, and/or larvae of the species A. diaperinus, and/or feces of the larvae of the species A. diaperinus.

In one embodiment, said material comprises or consist of at least one plastic polymer selected from the group consisting of polypropylene, polyurethane, polyester, polyacrylate, polyglycol, polyvinyl chloride, polycarbonate, polyvinylidene chloride, synthetic polyamide, polypropylene terephthalate, polyether and any mixes or copolymers thereof.

In one embodiment, said at least one plastic polymer is selected from the group consisting of polyurethane, polyester, polyacrylate, polyglycol, polyvinyl chloride, polyvinyl acetate, polycarbonate, polyvinylidene chloride, synthetic polyamide, polypropylene terephthalate, polyether and any mixes or copolymers thereof.

In one embodiment, said at least one plastic polymer is selected from the group consisting of polypropylene, polyurethane, polyacrylate, polyglycol, polyvinyl chloride, polycarbonate, polyvinylidene chloride, synthetic polyamide, polypropylene terephthalate, polyether and any mixes or copolymers thereof.

In one embodiment, said material comprises or consists of at least one plastic polymer selected from the group consisting of polyethylene terephthalate, polyurethane, polyester, polyacrylate, polyglycol, polyvinyl chloride, polycarbonate, polyvinylidene chloride, synthetic polyamide, polypropylene terephthalate, polyether and any mixes or copolymers thereof.

In one embodiment, said material comprises or consists of at least one plastic polymer selected from the group consisting of polyethylene terephthalate, polypropylene, polyester, polyacrylate, polyglycol, polyvinyl chloride, polycarbonate, polyvinylidene chloride, synthetic polyamide, polypropylene terephthalate, polyether and any mixes or copolymers thereof.

In one embodiment, said material comprises or consists of at least one plastic polymer selected from the group consisting of polyethylene terephthalate, polypropylene, polyurethane, polyacrylate, polyglycol, polyvinyl chloride, polycarbonate, polyvinylidene chloride, synthetic polyamide, polypropylene terephthalate, polyether and any mixes or copolymers thereof.

In one embodiment, said material comprises or consists of at least one plastic polymer selected from the group consisting of polyethylene terephthalate, polypropylene, polyurethane, polyester, polyglycol, polyvinyl chloride, polycarbonate, polyvinylidene chloride, synthetic polyamide, polypropylene terephthalate, polyether and any mixes or copolymers thereof.

In one embodiment, said material comprises or consists of at least one plastic polymer selected from the group consisting of polyethylene terephthalate, polypropylene, polyurethane, polyester, polyacrylate, polyglycol, polyvinyl chloride, polycarbonate, polyvinylidene chloride, synthetic polyamide, polypropylene terephthalate, polyether and any mixes or copolymers thereof.

In one embodiment, said material comprises or consists of at least one plastic polymer selected from the group consisting of polyethylene terephthalate, polypropylene, polyurethane, polyester, polyacrylate, polyvinyl chloride, polycarbonate, polyvinylidene chloride, synthetic polyamide, polypropylene terephthalate, polyether and any mixes or copolymers thereof.

In one embodiment, said material comprises or consists of at least one plastic polymer selected from the group consisting of polyethylene terephthalate, polypropylene, polyurethane, polyester, polyacrylate, polyglycol, polycarbonate, polyvinylidene chloride, synthetic polyamide, polypropylene terephthalate, polyether and any mixes or copolymers thereof.

In one embodiment, said material comprises or consists of at least one plastic polymer selected from the group consisting of polyethylene terephthalate, polypropylene, polyurethane, polyester, polyacrylate, polyglycol, polyvinyl chloride, polyvinylidene chloride, synthetic polyamide, polypropylene terephthalate, polyether and any mixes or copolymers thereof.

In one embodiment, said material comprises or consists of at least one plastic polymer selected from the group consisting of polyethylene terephthalate, polypropylene, polyurethane, polyester, polyacrylate, polyglycol, polyvinyl chloride, polycarbonate, synthetic polyamide, polypropylene terephthalate, polyether and any mixes or copolymers thereof.

In one embodiment, said material comprises or consists of at least one plastic polymer selected from the group consisting of polyethylene terephthalate, polypropylene, polyurethane, polyester, polyacrylate, polyglycol, polyvinyl chloride, polycarbonate, polyvinylidene chloride, polypropylene terephthalate, polyether and any mixes or copolymers thereof.

In one embodiment, said material comprises or consists of at least one plastic polymer selected from the group consisting of polyethylene terephthalate, polypropylene, polyurethane, polyester, polyacrylate, polyglycol, polyvinyl chloride, polycarbonate, polyvinylidene chloride, synthetic polyamide, polyether and any mixes or copolymers thereof.

In one embodiment, said at least one plastic polymer is selected from the group consisting of polyethylene terephthalate, polyester, polyacrylate, polyglycol, polyvinyl chloride, polycarbonate, polyvinylidene chloride, synthetic polyamide, polypropylene terephthalate, polyether and any mixes or copolymers thereof.

In one embodiment, said at least one plastic polymer is selected from the group consisting of polyethylene terephthalate, polyurethane, polyacrylate, polyglycol, polyvinyl chloride, polycarbonate, polyvinylidene chloride, synthetic polyamide, polypropylene terephthalate, polyether and any mixes or copolymers thereof.

In one embodiment, said at least one plastic polymer is selected from the group consisting of polyethylene terephthalate, polyurethane, polyester, polyglycol, polyvinyl chloride, polycarbonate, polyvinylidene chloride, synthetic polyamide, polypropylene terephthalate, polyether and any mixes or copolymers thereof.

In one embodiment, said at least one plastic polymer is selected from the group consisting of polyethylene terephthalate, polyurethane, polyester, polyacrylate, polyglycol, polyvinyl chloride, polycarbonate, polyvinylidene chloride, synthetic polyamide, polypropylene terephthalate, polyether and any mixes or copolymers thereof.

In one embodiment, said at least one plastic polymer is selected from the group consisting of polyethylene terephthalate, polyurethane, polyester, polyacrylate, polyvinyl chloride, polycarbonate, polyvinylidene chloride, synthetic polyamide, polypropylene terephthalate, polyether and any mixes or copolymers thereof.

In one embodiment, said at least one plastic polymer is selected from the group consisting of polyethylene terephthalate, polyurethane, polyester, polyacrylate, polyglycol, polycarbonate, polyvinylidene chloride, synthetic polyamide, polypropylene terephthalate, polyether and any mixes or copolymers thereof.

In one embodiment, said at least one plastic polymer is selected from the group consisting of polyethylene terephthalate, polyurethane, polyester, polyacrylate, polyglycol, polyvinyl chloride, polyvinylidene chloride, synthetic polyamide, polypropylene terephthalate, polyether and any mixes or copolymers thereof.

In one embodiment, said at least one plastic polymer is selected from the group consisting of polyethylene terephthalate, polyurethane, polyester, polyacrylate, polyglycol, polyvinyl chloride, polycarbonate, synthetic polyamide, polypropylene terephthalate, polyether and any mixes or copolymers thereof.

In one embodiment, said at least one plastic polymer is selected from the group consisting of polyethylene terephthalate, polyurethane, polyester, polyacrylate, polyglycol, polyvinyl chloride, polycarbonate, polyvinylidene chloride, polypropylene terephthalate, polyether and any mixes or copolymers thereof.

In one embodiment, said at least one plastic polymer is selected from the group consisting of polyethylene terephthalate, polyurethane, polyester, polyacrylate, polyglycol, polyvinyl chloride, polycarbonate, polyvinylidene chloride, synthetic polyamide, polyether and any mixes or copolymers thereof.

In one embodiment, said at least one plastic polymer is selected from the group consisting of polyurethane, polyacrylate, polyglycol, polyvinyl chloride, polycarbonate, polyvinylidene chloride, synthetic polyamide, polypropylene terephthalate, polyether and any mixes or copolymers thereof.

In one embodiment, said at least one plastic polymer is selected from the group consisting of polyethylene terephthalate, polyurethane, polyacrylate, polyglycol, polyvinyl chloride, polycarbonate, polyvinylidene chloride, synthetic polyamide, polypropylene terephthalate, polyether and any mixes or copolymers thereof.

In one embodiment, said at least one plastic polymer is selected from the group consisting of polyethylene terephthalate, polyacrylate, polyglycol, polyvinyl chloride, polycarbonate, polyvinylidene chloride, synthetic polyamide, polypropylene terephthalate, polyether and any mixes or copolymers thereof.

In one embodiment, said at least one plastic polymer is selected from the group consisting of polyethylene terephthalate, polyurethane, polyacrylate, polyglycol, polyvinyl chloride, polycarbonate, polyvinylidene chloride, synthetic polyamide, polypropylene terephthalate, polyether and any mixes or copolymers thereof.

In one embodiment, said at least one plastic polymer is selected from the group consisting of polyethylene terephthalate, polyurethane, polyglycol, polyvinyl chloride, polycarbonate, polyvinylidene chloride, synthetic polyamide, polypropylene terephthalate, polyether and any mixes or copolymers thereof.

In one embodiment, said at least one plastic polymer is selected from the group consisting of polyethylene terephthalate, polyurethane, polyacrylate, polyglycol, polyvinyl chloride, polycarbonate, polyvinylidene chloride, synthetic polyamide, polypropylene terephthalate, polyether and any mixes or copolymers thereof.

In one embodiment, said at least one plastic polymer is selected from the group consisting of polyethylene terephthalate, polyurethane, polyacrylate, polyvinyl chloride, polycarbonate, polyvinylidene chloride, synthetic polyamide, polypropylene terephthalate, polyether and any mixes or copolymers thereof.

In one embodiment, said at least one plastic polymer is selected from the group consisting of polyethylene terephthalate, polyurethane, polyacrylate, polyglycol, polycarbonate, polyvinylidene chloride, synthetic polyamide, polypropylene terephthalate, polyether and any mixes or copolymers thereof.

In one embodiment, said at least one plastic polymer is selected from the group consisting of polyethylene terephthalate, polyurethane, polyacrylate, polyglycol, polyvinyl chloride, polyvinylidene chloride, synthetic polyamide, polypropylene terephthalate, polyether and any mixes or copolymers thereof.

In one embodiment, said at least one plastic polymer is selected from the group consisting of polyethylene terephthalate, polyurethane, polyacrylate, polyglycol, polyvinyl chloride, polycarbonate, synthetic polyamide, polypropylene terephthalate, polyether and any mixes or copolymers thereof.

In one embodiment, said at least one plastic polymer is selected from the group consisting of polyethylene terephthalate, polyurethane, polyacrylate, polyglycol, polyvinyl chloride, polycarbonate, polyvinylidene chloride, polypropylene terephthalate, polyether and any mixes or copolymers thereof.

In one embodiment, said at least one plastic polymer is selected from the group consisting of polyethylene terephthalate, polyurethane, polyacrylate, polyglycol, polyvinyl chloride, polycarbonate, polyvinylidene chloride, synthetic polyamide, polyether and any mixes or copolymers thereof.

In one embodiment, said material comprises or consists of at least one plastic polymer selected from the group consisting of polypropylene terephthalate and polyether.

In one embodiment, said polymer is selected from the group consisting of polyurethane, polyester, polyacrylate, polyglycol, polyvinyl chloride, polycarbonate, polyvinylidene chloride, synthetic polyamide, polypropylene terephtalate, polyether and any mixes or copolymers thereof;
or the group consisting of polypropylene, polyester, polyacrylate, polyglycol, polyvinyl chloride, polycarbonate, polyvinylidene chloride, synthetic polyamide, polypropylene terephtalate, polyether and any mixes or copolymers thereof;
or the group consisting of polypropylene, polyurethane, polyacrylate, polyglycol, polyvinyl chloride, polycarbonate, polyvinylidene chloride, synthetic polyamide, polypropylene terephtalate, polyether and any mixes or copolymers thereof;
or the group consisting of polypropylene, polyurethane, polyester, polyglycol, polyvinyl chloride, polycarbonate, polyvinylidene chloride, synthetic polyamide, polypropylene terephtalate, polyether and any mixes or copolymers thereof;
or the group consisting of polypropylene, polyurethane, polyester, polyacrylate, polyvinyl chloride, polycarbonate, polyvinylidene chloride, synthetic polyamide, polypropylene terephtalate, polyether and any mixes or copolymers thereof;
or the group consisting of polypropylene, polyurethane, polyester, polyacrylate, polyglycol, polycarbonate, polyvinylidene chloride, synthetic polyamide, polypropylene terephtalate, polyether and any mixes or copolymers thereof;
or the group consisting of polypropylene, polyurethane, polyester, polyacrylate, polyglycol, polyvinyl chloride, polyvinylidene chloride, synthetic polyamide, polypropylene terephtalate, polyether and any mixes or copolymers thereof;
or the group consisting of polypropylene, polyurethane, polyester, polyacrylate, polyglycol, polyvinyl chloride, polycarbonate, synthetic polyamide, polypropylene terephtalate, polyether and any mixes or copolymers thereof;
or the group consisting of polypropylene, polyurethane, polyester, polyacrylate, polyglycol, polyvinyl chloride, polycarbonate, polyvinylidene chloride, polypropylene terephtalate, polyether and any mixes or copolymers thereof;
or the group consisting of polypropylene, polyurethane, polyester, polyacrylate, polyglycol, polyvinyl chloride, polycarbonate, polyvinylidene chloride, synthetic polyamide, polyether and any mixes or copolymers thereof; or the group consisting of polypropylene, polyurethane, polyester, polyacrylate, polyglycol, polyvinyl chloride, polycarbonate, polyvinylidene chloride, synthetic polyamide, polypropylene terephtalate, and any mixes or copolymers thereof;
or the group consisting of polyethylene terephthalate, polyester, polyacrylate, polyglycol, polyvinyl chloride, polycarbonate, polyvinylidene chloride, synthetic polyamide, polypropylene terephtalate, polyether and any mixes or copolymers thereof;
or the group consisting of polyethylene terephthalate, polyurethane, polyacrylate, polyglycol, polyvinyl chloride, polycarbonate, polyvinylidene chloride, synthetic polyamide, polypropylene terephtalate, polyether and any mixes or copolymers thereof;
or the group consisting of polyethylene terephthalate, polyurethane, polyester, polyglycol, polyvinyl chloride, polycarbonate, polyvinylidene chloride, synthetic polyamide, polypropylene terephtalate, polyether and any mixes or copolymers thereof;
or the group consisting of polyethylene terephthalate, polyurethane, polyester, polyacrylate, polyvinyl chloride, polycarbonate, polyvinylidene chloride, synthetic polyamide, polypropylene terephtalate, polyether and any mixes or copolymers thereof;
or the group consisting of polyethylene terephthalate, polyurethane, polyester, polyacrylate, polyglycol, polycarbonate, polyvinylidene chloride, synthetic polyamide, polypropylene terephtalate, polyether and any mixes or copolymers thereof;
or the group consisting of polyethylene terephthalate, polyurethane, polyester, polyacrylate, polyglycol, polyvinyl chloride, polyvinylidene chloride, synthetic polyamide, polypropylene terephtalate, polyether and any mixes or copolymers thereof;
or the group consisting of polyethylene terephthalate, polyurethane, polyester, polyacrylate, polyglycol, polyvinyl chloride, polycarbonate, synthetic polyamide, polypropylene terephtalate, polyether and any mixes or copolymers thereof;
or the group consisting of polyethylene terephthalate, polyurethane, polyester, polyacrylate, polyglycol, polyvinyl chloride, polycarbonate, polyvinylidene chloride, polypropylene terephtalate, polyether and any mixes or copolymers thereof;
or the group consisting of polyethylene terephthalate, polyurethane, polyester, polyacrylate, polyglycol, polyvinyl chloride, polycarbonate, polyvinylidene chloride, synthetic polyamide, polyether and any mixes or copolymers thereof;
or the group consisting of polyethylene terephthalate, polyurethane, polyester, polyacrylate, polyglycol, polyvinyl chloride, polycarbonate, polyvinylidene chloride, synthetic polyamide, polypropylene terephtalate, and any mixes or copolymers thereof;
or the group consisting of polyethylene terephthalate, polypropylene, polyacrylate, polyglycol, polyvinyl chloride, polycarbonate, polyvinylidene chloride, synthetic polyamide, polypropylene terephthalate, polyether and any mixes or copolymers thereof;
or the group consisting of polyethylene terephthalate, polypropylene, polyester, polyglycol, polyvinyl chloride, polycarbonate, polyvinylidene chloride, synthetic polyamide, polypropylene terephtalate, polyether and any mixes or copolymers thereof;
or the group consisting of polyethylene terephthalate, polypropylene, polyester, polyacrylate, polyvinyl chloride, polycarbonate, polyvinylidene chloride, synthetic polyamide, polypropylene terephtalate, polyether and any mixes or copolymers thereof;
or the group consisting of polyethylene terephthalate, polypropylene, polyester, polyacrylate, polyglycol, polycarbonate, polyvinylidene chloride, synthetic polyamide, polypropylene terephtalate, polyether and any mixes or copolymers thereof;
or the group consisting of polyethylene terephthalate, polypropylene, polyester, polyacrylate, polyglycol, polyvinyl chloride, polyvinylidene chloride, synthetic polyamide, polypropylene terephtalate, polyether and any mixes or copolymers thereof;
or the group consisting of polyethylene terephthalate, polypropylene, polyester, polyacrylate, polyglycol, polyvinyl chloride, polycarbonate, synthetic polyamide, polypropylene terephtalate, polyether and any mixes or copolymers thereof;
or the group consisting of polyethylene terephthalate, polypropylene, polyester, polyacrylate, polyglycol, polyvinyl chloride, polycarbonate, polyvinylidene chloride, polypropylene terephtalate, polyether and any mixes or copolymers thereof;
or the group consisting of polyethylene terephthalate, polypropylene, polyester, polyacrylate, polyglycol, polyvinyl chloride, polycarbonate, polyvinylidene chloride, synthetic polyamide, polyether and any mixes or copolymers thereof;
or the group consisting of polyethylene terephthalate, polypropylene, polyester, polyacrylate, polyglycol, polyvinyl chloride, polycarbonate, polyvinylidene chloride, synthetic polyamide, polypropylene terephtalate and any mixes or copolymers thereof;
or the group consisting of polyethylene terephthalate, polypropylene, polyurethane, polyglycol, polyvinyl chloride, polycarbonate, polyvinylidene chloride, synthetic polyamide, polypropylene terephtalate, polyether and any mixes or copolymers thereof;
or the group consisting of polyethylene terephthalate, polypropylene, polyurethane, polyacrylate, polyvinyl chloride, polycarbonate, polyvinylidene chloride, synthetic polyamide, polypropylene terephtalate, polyether and any mixes or copolymers thereof;
or the group consisting of polyethylene terephthalate, polypropylene, polyurethane, polyacrylate, polyglycol, polycarbonate, polyvinylidene chloride, synthetic polyamide, polypropylene terephtalate, polyether and any mixes or copolymers thereof;
or the group consisting of polyethylene terephthalate, polypropylene, polyurethane, polyacrylate, polyglycol, polyvinyl chloride, polyvinylidene chloride, synthetic polyamide, polypropylene terephtalate, polyether and any mixes or copolymers thereof;
or the group consisting of polyethylene terephthalate, polypropylene, polyurethane, polyacrylate, polyglycol, polyvinyl chloride, polycarbonate, synthetic polyamide, polypropylene terephtalate, polyether and any mixes or copolymers thereof;
or the group consisting of polyethylene terephthalate, polypropylene, polyurethane, polyacrylate, polyglycol, polyvinyl chloride, polycarbonate, polyvinylidene chloride, polypropylene terephtalate, polyether and any mixes or copolymers thereof;
or the group consisting of polyethylene terephthalate, polypropylene, polyurethane, polyacrylate, polyglycol, polyvinyl chloride, polycarbonate, polyvinylidene chloride, synthetic polyamide, polyether and any mixes or copolymers thereof;
or the group consisting of polyethylene terephthalate, polypropylene, polyurethane, polyacrylate, polyglycol, polyvinyl chloride, polycarbonate, polyvinylidene chloride, synthetic polyamide, polypropylene terephtalate and any mixes or copolymers thereof;
or the group consisting of polyethylene terephthalate, polypropylene, polyurethane, polyester, polyvinyl chloride, polycarbonate, polyvinylidene chloride, synthetic polyamide, polypropylene terephtalate, polyether and any mixes or copolymers thereof;
or the group consisting of polyethylene terephthalate, polypropylene, polyurethane, polyester, polyglycol, polycarbonate, polyvinylidene chloride, synthetic polyamide, polypropylene terephtalate, polyether and any mixes or copolymers thereof;
or the group consisting of polyethylene terephthalate, polypropylene, polyurethane, polyester, polyglycol, polyvinyl chloride, polyvinylidene chloride synthetic polyamide, polypropylene terephtalate, polyether and any mixes or copolymers thereof;
or the group consisting of polyethylene terephthalate, polypropylene, polyurethane, polyester, polyglycol, polyvinyl chloride, polycarbonate, synthetic polyamide, polypropylene terephtalate, polyether and any mixes or copolymers thereof;
or the group consisting of polyethylene terephthalate, polypropylene, polyurethane, polyester, polyglycol, polyvinyl chloride, polycarbonate, polyvinylidene chloride, polypropylene terephtalate, polyether and any mixes or copolymers thereof;
or the group consisting of polyethylene terephthalate, polypropylene, polyurethane, polyester, polyacrylate, polyglycol, polyvinyl chloride, polycarbonate, polyvinylidene chloride, synthetic polyamide, polyether and any mixes or copolymers thereof;
or the group consisting of polyethylene terephthalate, polypropylene, polyurethane, polyester, polyacrylate, polyglycol, polyvinyl chloride, polycarbonate, polyvinylidene chloride, synthetic polyamide, polypropylene terephtalate and any mixes or copolymers thereof;
or the group consisting of polyethylene terephthalate, polypropylene, polyurethane, polyester, polyacrylate, polycarbonate, polyvinylidene chloride, synthetic polyamide, polypropylene terephtalate, polyether and any mixes or copolymers thereof;
or the group consisting of polyethylene terephthalate, polypropylene, polyurethane, polyester, polyacrylate, polyvinyl chloride, polyvinylidene chloride, synthetic polyamide, polypropylene terephtalate, polyether and any mixes or copolymers thereof;
or the group consisting of polyethylene terephthalate, polypropylene, polyurethane, polyester, polyacrylate, polyvinyl chloride, polycarbonate, synthetic polyamide, polypropylene terephtalate, polyether and any mixes or copolymers thereof;
or the group consisting of polyethylene terephthalate, polypropylene, polyurethane, polyester, polyacrylate, polyvinyl chloride, polycarbonate, polyvinylidene chloride, polypropylene terephtalate, polyether and any mixes or copolymers thereof;
or the group consisting of polyethylene terephthalate, polypropylene, polyurethane, polyester, polyacrylate, polyvinyl chloride, polycarbonate, polyvinylidene chloride, synthetic polyamide, polyether and any mixes or copolymers thereof;
or the group consisting of polyethylene terephthalate, polypropylene, polyurethane, polyester, polyacrylate, polyvinyl chloride, polycarbonate, polyvinylidene chloride, synthetic polyamide, polypropylene terephtalate and any mixes or copolymers thereof;
or the group consisting of polyethylene terephthalate, polypropylene, polyurethane, polyester, polyacrylate, polyglycol, polyvinylidene chloride, synthetic polyamide, polypropylene terephtalate, polyether and any mixes or copolymers thereof;
or the group consisting of polyethylene terephthalate, polypropylene, polyurethane, polyester, polyacrylate, polyglycol, polycarbonate, synthetic polyamide, polypropylene terephtalate, polyether and any mixes or copolymers thereof;
or the group consisting of polyethylene terephthalate, polypropylene, polyurethane, polyester, polyacrylate, polyglycol, polycarbonate, polyvinylidene chloride, polypropylene terephtalate, polyether and any mixes or copolymers thereof;
or the group consisting of polyethylene terephthalate, polypropylene, polyurethane, polyester, polyacrylate, polyglycol, polycarbonate, polyvinylidene chloride, synthetic polyamide, polyether and any mixes or copolymers thereof;
or the group consisting of polyethylene terephthalate, polypropylene, polyurethane, polyester, polyacrylate, polyglycol, polycarbonate, polyvinylidene chloride, synthetic polyamide, polypropylene terephtalate and any mixes or copolymers thereof;
or the group consisting of polyethylene terephthalate, polypropylene, polyurethane, polyester, polyacrylate, polyglycol, polyvinyl chloride, synthetic polyamide, polypropylene terephtalate, polyether and any mixes or copolymers thereof;
or the group consisting of polyethylene terephthalate, polypropylene, polyurethane, polyester, polyacrylate, polyglycol, polyvinyl chloride, polyvinylidene chloride, polypropylene terephtalate, polyether and any mixes or copolymers thereof;
or the group consisting of polyethylene terephthalate, polypropylene, polyurethane, polyester, polyacrylate, polyglycol, polyvinyl chloride, polyvinylidene chloride, synthetic polyamide, polyether and any mixes or copolymers thereof;
or the group consisting of polyethylene terephthalate, polypropylene, polyurethane, polyester, polyacrylate, polyglycol, polyvinyl chloride, polyvinylidene chloride, synthetic polyamide, polypropylene terephtalate and any mixes or copolymers thereof;
or the group consisting of polyethylene terephthalate, polypropylene, polyurethane, polyester, polyacrylate, polyglycol, polyvinyl chloride, polycarbonate, polypropylene terephtalate, polyether and any mixes or copolymers thereof;
or the group consisting of polyethylene terephthalate, polypropylene, polyurethane, polyester, polyacrylate, polyglycol, polyvinyl chloride, polycarbonate, synthetic polyamide, polyether and any mixes or copolymers thereof;
or the group consisting of polyethylene terephthalate, polypropylene, polyurethane, polyester, polyacrylate, polyglycol, polyvinyl chloride, polycarbonate, synthetic polyamide, polypropylene terephtalate and any mixes or copolymers thereof;
or the group consisting of polyethylene terephthalate, polypropylene, polyurethane, polyester, polyacrylate, polyglycol, polyvinyl chloride, polycarbonate, polyvinylidene chloride, polyether and any mixes or copolymers thereof;
or the group consisting of polyethylene terephthalate, polypropylene, polyurethane, polyester, polyacrylate, polyglycol, polyvinyl chloride, polycarbonate, polyvinylidene chloride, polypropylene terephtalate and any mixes or copolymers thereof;
or the group consisting of polyethylene terephthalate, polypropylene, polyurethane, polyester, polyacrylate, polyglycol, polyvinyl chloride, polycarbonate, polyvinylidene chloride, synthetic polyamide and any mixes or copolymers thereof.

In one particular embodiment, the polymer is selected from the group consisting of polypropylene, polyurethane, synthetic polyamide, polyester, polyacrylate, and any mixes or copolymers thereof.

In one embodiment, the polymer is selected from the group consisting of polypropylene, polyurethane, synthetic polyamide, polyester, and any mixes or copolymers thereof. In one particular embodiment, the polymer is selected from the group consisting of polyurethane, synthetic polyamide, polyester, polyvinyl acetate, polyacrylate, and any mixes or copolymers thereof.

In one embodiment, the polymer is selected from the group consisting of polyurethane, synthetic polyamide, polyester, polyvinyl acetate and any mixes or copolymers thereof.

In one particular embodiment, the polymer is selected from the group consisting of polyurethane, synthetic polyamide, polyester, polyacrylate, and any mixes or copolymers thereof.

In one embodiment, the polymer is selected from the group consisting of polyurethane, synthetic polyamide, polyester, and any mixes or copolymers thereof.

In another embodiment, said at least one plastic polymer is selected from the group consisting of polyethylene terephthalate, polypropylene terephthalate, polyether, polyurethane, polyacrylate, polyglycol, polyvinyl chloride, polycarbonate, polyvinylidene chloride, polyacrylonitrile, synthetic polyamide and any mixes or copolymers thereof, and any copolymers of polyethylene;
or the group consisting of polyethylene terephthalate, polypropylene terephthalate, polyether, polyurethane, polyacrylate, polyglycol, polyvinyl chloride, polycarbonate, polyvinylidene chloride, polyacrylonitrile, synthetic polyamide and any mixes or copolymers thereof;
or the group consisting of polyethylene terephthalate, polypropylene terephthalate, polyether, polyurethane, polyacrylate, polyglycol, polyvinyl chloride, polycarbonate, polyvinylidene chloride, synthetic polyamide and any mixes or copolymers thereof, and any copolymers of polyethylene;
or the group consisting of polyethylene terephthalate, polypropylene terephthalate, polyether, polyurethane, polyacrylate, polyglycol, polyvinyl chloride, polycarbonate, polyvinylidene chloride, synthetic polyamide and any mixes or copolymers thereof, and any copolymers of polyacrylonitrile;
or the group consisting of polyethylene terephthalate, polypropylene terephthalate, polyether, polyurethane, polyacrylate, polyglycol, polyvinyl chloride, polycarbonate, polyvinylidene chloride, synthetic polyamide and any mixes or copolymers thereof, and any copolymers of polyethylene and polyacrylonitrile;
or the group consisting of polyethylene terephthalate, polypropylene terephthalate, polyether, polyurethane, polyacrylate, polyglycol, polyvinyl chloride, polycarbonate, polyvinylidene chloride, polyacrylonitrile, synthetic polyamide and any mixes or copolymers thereof, and poly(ethylene-vinyl acetate) copolymer;
or the group consisting of polyethylene terephthalate, polypropylene terephthalate, polyether, polyurethane, polyacrylate, polyglycol, polyvinyl chloride, polycarbonate, polyvinylidene chloride, synthetic polyamide and any mixes or copolymers thereof, and poly(ethylene-vinyl acetate) copolymer;
or the group consisting of polyethylene terephthalate, polypropylene terephthalate, polyether, polyurethane, polyacrylate, polyglycol, polyvinyl chloride, polycarbonate, polyvinylidene chloride, synthetic polyamide and any mixes or copolymers thereof, poly(ethylene-vinyl acetate) copolymer, and any copolymers of polyacrylonitrile;
or the group consisting of polyethylene terephthalate, polypropylene terephthalate, polyether, polyurethane, polyacrylate, polyglycol, polyvinyl chloride, polycarbonate, polyvinylidene chloride, synthetic polyamide and any mixes or copolymers thereof, poly(acrylonitrile-butadiene) copolymer and any copolymers of polyethylene;
or the group consisting of polyethylene terephthalate, polypropylene terephthalate, polyether, polyurethane, polyacrylate, polyglycol, polyvinyl chloride, polycarbonate, polyvinylidene chloride, synthetic polyamide and any mixes or copolymers thereof, poly(acrylonitrile-butadiene) copolymer and poly(ethylene-vinyl acetate) copolymer;
or the group consisting of polyethylene terephthalate, polypropylene terephthalate, polyether, polyurethane, polyacrylate, polyglycol, polyvinyl chloride, polycarbonate, polyvinylidene chloride, synthetic polyamide and any mixes or copolymers thereof, and poly(acrylonitrile-butadiene) copolymer.

In another embodiment, said at least one plastic polymer is selected from the group consisting of polyurethane, synthetic polyamide, polyacrylate, polyvinyl chloride, polyacrylonitrile, and any mixes or copolymers thereof, and any copolymers of polyethylene;
or the group consisting of polyurethane, synthetic polyamide, polyacrylate, polyvinyl chloride, polyacrylonitrile, and any mixes or copolymers thereof, and poly(ethylene-vinyl acetate) copolymer;
or the group consisting of polyurethane, synthetic polyamide, polyacrylate, polyvinyl chloride, and any mixes or copolymers thereof, and any copolymers of polyethylene and polyacrylonitrile;
or the group consisting of polyurethane, synthetic polyamide, polyacrylate, polyvinyl chloride, and any mixes or copolymers thereof, poly(acrylonitrile-butadiene) copolymer and any copolymers of polyethylene;
or the group consisting of polyurethane, synthetic polyamide, polyacrylate, polyvinyl chloride, and any mixes or copolymers thereof, poly(ethylene-vinyl acetate) copolymer, and any copolymers of polyacrylonitrile;
or the group consisting of polyurethane, synthetic polyamide, polyacrylate, polyvinyl chloride, and any mixes or copolymers thereof, poly(acrylonitrile-butadiene) copolymer and poly(ethylene-vinyl acetate) copolymer;
or the group consisting of polyurethane, synthetic polyamide, polyacrylate, polyacrylonitrile, and any mixes or copolymers thereof, and any copolymers of polyethylene;
or the group consisting of polyurethane, synthetic polyamide, polyacrylate, polyacrylonitrile, and any mixes or copolymers thereof, and poly(ethylene-vinyl acetate) copolymer;
or the group consisting of polyurethane, synthetic polyamide, polyacrylate, and any mixes or copolymers thereof, and any copolymers of polyethylene and polyacrylonitrile;
or the group consisting of polyurethane, synthetic polyamide, polyacrylate, and any mixes or copolymers thereof, poly(acrylonitrile-butadiene) copolymer and any copolymers of polyethylene;
or the group consisting of polyurethane, synthetic polyamide, polyacrylate, and any mixes or copolymers thereof, poly(ethylene-vinyl acetate) copolymer, and any copolymers of polyacrylonitrile;
or the group consisting of polyurethane, synthetic polyamide, polyacrylate, and any mixes or copolymers thereof, poly(acrylonitrile-butadiene) copolymer and poly(ethylene-vinyl acetate) copolymer;
or the group consisting of polyurethane, synthetic polyamide, polyacrylate, polyvinyl chloride, polyacrylonitrile, and any mixes or copolymers thereof;
or the group consisting of polyurethane, synthetic polyamide, polyacrylate, polyvinyl chloride, and any mixes or copolymers thereof, and any copolymers of polyacrylonitrile;
or the group consisting of polyurethane, synthetic polyamide, polyacrylate, polyvinyl chloride, and any mixes or copolymers thereof, poly(acrylonitrile-butadiene) copolymer;
or the group consisting of polyurethane, synthetic polyamide, polyacrylate, polyvinyl chloride, and any mixes or copolymers thereof, and any copolymers of polyethylene;
or the group consisting of polyurethane, synthetic polyamide, polyacrylate, polyvinyl chloride, and any mixes or copolymers thereof, and poly(ethylene-vinyl acetate) copolymer;
or the group consisting of polyurethane, synthetic polyamide, polyacrylate, polyvinyl chloride, and any mixes or copolymers thereof;
or the group consisting of polyurethane, synthetic polyamide, polyacrylate, polyacrylonitrile, and any mixes or copolymers thereof;
or the group consisting of polyurethane, synthetic polyamide, polyacrylate, and any mixes or copolymers thereof, and any copolymers of polyethylene;
or the group consisting of polyurethane, synthetic polyamide, polyacrylate, and any mixes or copolymers thereof, and any copolymers of polyacrylonitrile;
or the group consisting of polyurethane, synthetic polyamide, polyacrylate, and any mixes or copolymers thereof, and poly(acrylonitrile-butadiene) copolymer;
or the group consisting of polyurethane, synthetic polyamide, polyacrylate, and any mixes or copolymers thereof, and poly(ethylene-vinyl acetate) copolymer.

In another embodiment, said at least one plastic polymer is selected from the group consisting of polyurethane, synthetic polyamide, polyvinyl chloride, polyacrylonitrile, and any mixes or copolymers thereof, and any copolymers of polyethylene;
or the group consisting of polyurethane, synthetic polyamide, polyvinyl chloride, polyacrylonitrile, and any mixes or copolymers thereof, and poly(ethylene-vinyl acetate) copolymer;
or the group consisting of polyurethane, synthetic polyamide, polyvinyl chloride, and any mixes or copolymers thereof, and any copolymers of polyethylene and polyacrylonitrile;
or the group consisting of polyurethane, synthetic polyamide, polyvinyl chloride, and any mixes or copolymers thereof, poly(acrylonitrile-butadiene) copolymer and any copolymers of polyethylene;
or the group consisting of polyurethane, synthetic polyamide, polyvinyl chloride, and any mixes or copolymers thereof, poly(ethylene-vinyl acetate) copolymer, and any copolymers of polyacrylonitrile;
or the group consisting of polyurethane, synthetic polyamide, polyvinyl chloride, and any mixes or copolymers thereof, poly(acrylonitrile-butadiene) copolymer and poly(ethylene-vinyl acetate) copolymer;
or the group consisting of polyurethane, synthetic polyamide, polyacrylonitrile, and any mixes or copolymers thereof, and any copolymers of polyethylene;
or the group consisting of polyurethane, synthetic polyamide, polyacrylonitrile, and any mixes or copolymers thereof, and poly(ethylene-vinyl acetate) copolymer;
or the group consisting of polyurethane, synthetic polyamide, and any mixes or copolymers thereof, and any copolymers of polyethylene and polyacrylonitrile;
or the group consisting of polyurethane, synthetic polyamide, and any mixes or copolymers thereof, poly(acrylonitrile-butadiene) copolymer and any copolymers of polyethylene;
or the group consisting of polyurethane, synthetic polyamide, and any mixes or copolymers thereof, poly(ethylene-vinyl acetate) copolymer, and any copolymers of polyacrylonitrile;
or the group consisting of polyurethane, synthetic polyamide, and any mixes or copolymers thereof, poly(acrylonitrile-butadiene) copolymer and poly(ethylene-vinyl acetate) copolymer;
or the group consisting of polyurethane, synthetic polyamide, polyvinyl chloride, polyacrylonitrile, and any mixes or copolymers thereof;
or the group consisting of polyurethane, synthetic polyamide, polyvinyl chloride, and any mixes or copolymers thereof, and any copolymers of polyacrylonitrile;
or the group consisting of polyurethane, synthetic polyamide, polyvinyl chloride, and any mixes or copolymers thereof, poly(acrylonitrile-butadiene) copolymer;
or the group consisting of polyurethane, synthetic polyamide, polyvinyl chloride, and any mixes or copolymers thereof, and any copolymers of polyethylene;
or the group consisting of polyurethane, synthetic polyamide, polyvinyl chloride, and any mixes or copolymers thereof, and poly(ethylene-vinyl acetate) copolymer;
or the group consisting of polyurethane, synthetic polyamide, polyvinyl chloride, and any mixes or copolymers thereof;
or the group consisting of polyurethane, synthetic polyamide, polyacrylonitrile, and any mixes or copolymers thereof;
or the group consisting of polyurethane, synthetic polyamide, and any mixes or copolymers thereof, and any copolymers of polyethylene;
or the group consisting of polyurethane, synthetic polyamide, and any mixes or copolymers thereof, and any copolymers of polyacrylonitrile;
or the group consisting of polyurethane, synthetic polyamide, and any mixes or copolymers thereof, and poly(acrylonitrile-butadiene) copolymer;
or the group consisting of polyurethane, synthetic polyamide, and any mixes or copolymers thereof, and poly(ethylene-vinyl acetate) copolymer.

In another embodiment, said at least one plastic polymer is selected from the group consisting of polyurethane, polyacrylate, polyvinyl chloride, polyacrylonitrile, and any mixes or copolymers thereof, and any copolymers of polyethylene;
or the group consisting of polyurethane, polyacrylate, polyvinyl chloride, polyacrylonitrile, and any mixes or copolymers thereof, and poly(ethylene-vinyl acetate) copolymer;
or the group consisting of polyurethane, polyacrylate, polyvinyl chloride, and any mixes or copolymers thereof, and any copolymers of polyethylene and copolymers of polyacrylonitrile;
or the group consisting of polyurethane, polyacrylate, polyvinyl chloride, and any mixes or copolymers thereof, poly(acrylonitrile-butadiene) copolymer and any copolymers of polyethylene;
or the group consisting of polyurethane, polyacrylate, polyvinyl chloride, and any mixes or copolymers thereof, poly(ethylene-vinyl acetate) copolymer, and any copolymers of polyacrylonitrile;
or the group consisting of polyurethane, polyacrylate, polyvinyl chloride, and any mixes or copolymers thereof, poly(acrylonitrile-butadiene) copolymer and poly(ethylene-vinyl acetate) copolymer;
or the group consisting of polyurethane, polyacrylate, polyacrylonitrile, and any mixes or copolymers thereof, and any copolymers of polyethylene;
or the group consisting of polyurethane, polyacrylate, polyacrylonitrile, and any mixes or copolymers thereof, and poly(ethylene-vinyl acetate) copolymer;
or the group consisting of polyurethane, polyacrylate, and any mixes or copolymers thereof, and any copolymers of polyethylene and copolymers of polyacrylonitrile;
or the group consisting of polyurethane, polyacrylate, and any mixes or copolymers thereof, poly(acrylonitrile-butadiene) copolymer and any copolymers of polyethylene;
or the group consisting of polyurethane, polyacrylate, and any mixes or copolymers thereof, poly(ethylene-vinyl acetate) copolymer, and any copolymers of polyacrylonitrile;
or the group consisting of polyurethane, polyacrylate, and any mixes or copolymers thereof, poly(acrylonitrile-butadiene) copolymer and poly(ethylene-vinyl acetate) copolymer;
or the group consisting of polyurethane, polyacrylate, polyvinyl chloride, polyacrylonitrile, and any mixes or copolymers thereof;
or the group consisting of polyurethane, polyacrylate, polyvinyl chloride, and any mixes or copolymers thereof, and any copolymers of polyacrylonitrile;
or the group consisting of polyurethane, polyacrylate, polyvinyl chloride, and any mixes or copolymers thereof, poly(acrylonitrile-butadiene) copolymer;
or the group consisting of polyurethane, polyacrylate, polyvinyl chloride, and any mixes or copolymers thereof, and any copolymers of polyethylene;
or the group consisting of polyurethane, polyacrylate, polyvinyl chloride, and any mixes or copolymers thereof, and poly(ethylene-vinyl acetate) copolymer;
or the group consisting of polyurethane, polyacrylate, polyvinyl chloride, and any mixes or copolymers thereof;
or the group consisting of polyurethane, polyacrylate, polyacrylonitrile, and any mixes or copolymers thereof;
or the group consisting of polyurethane, polyacrylate, and any mixes or copolymers thereof, and any copolymers of polyethylene;
or the group consisting of polyurethane, polyacrylate, and any mixes or copolymers thereof, and any copolymers of polyacrylonitrile;
or the group consisting of polyurethane, polyacrylate, and any mixes or copolymers thereof, and poly(acrylonitrile-butadiene) copolymer;
or the group consisting of polyurethane, polyacrylate, and any mixes or copolymers thereof, and poly(ethylene-vinyl acetate) copolymer.

In another embodiment, said at least one plastic polymer is selected from the group consisting of polyurethane, polyvinyl chloride, polyacrylonitrile, and any mixes or copolymers thereof, and any copolymers of polyethylene;
or the group consisting of polyurethane, polyvinyl chloride, polyacrylonitrile, and any mixes or copolymers thereof, and poly(ethylene-vinyl acetate) copolymer;
or the group consisting of polyurethane, polyvinyl chloride, and any mixes or copolymers thereof, and any copolymers of polyethylene and copolymers of polyacrylonitrile;
or the group consisting of polyurethane, polyvinyl chloride, and any mixes or copolymers thereof, poly(acrylonitrile-butadiene) copolymer and any copolymers of polyethylene;
or the group consisting of polyurethane, polyvinyl chloride, and any mixes or copolymers thereof, poly(ethylene-vinyl acetate) copolymer, and any copolymers of polyacrylonitrile;
or the group consisting of polyurethane, polyvinyl chloride, and any mixes or copolymers thereof, poly(acrylonitrile-butadiene) copolymer and poly(ethylene-vinyl acetate) copolymer;
or the group consisting of polyurethane, polyacrylonitrile, and any mixes or copolymers thereof, and any copolymers of polyethylene;
or the group consisting of polyurethane, polyacrylonitrile, and any mixes or copolymers thereof, and poly(ethylene-vinyl acetate) copolymer;
or the group consisting of polyurethane, and any mixes or copolymers thereof, and any copolymers of polyethylene and copolymers of polyacrylonitrile;
or the group consisting of polyurethane, and any mixes or copolymers thereof, poly(acrylonitrile-butadiene) copolymer and any copolymers of polyethylene;
or the group consisting of polyurethane, and any mixes or copolymers thereof, poly(ethylene-vinyl acetate) copolymer, and any copolymers of polyacrylonitrile;
or the group consisting of polyurethane, and any mixes or copolymers thereof, poly(acrylonitrile-butadiene) copolymer and poly(ethylene-vinyl acetate) copolymer;
or the group consisting of polyurethane, polyvinyl chloride, polyacrylonitrile, and any mixes or copolymers thereof;
or the group consisting of polyurethane, polyvinyl chloride, and any mixes or copolymers thereof, and any copolymers of polyacrylonitrile;
or the group consisting of polyurethane, polyvinyl chloride, and any mixes or copolymers thereof, and poly(acrylonitrile-butadiene) copolymer;
or the group consisting of polyurethane, polyvinyl chloride, and any mixes or copolymers thereof, and any copolymers of polyethylene;
or the group consisting of polyurethane, polyvinyl chloride, and any mixes or copolymers thereof, and poly(ethylene-vinyl acetate) copolymer;
or the group consisting of polyurethane, polyvinyl chloride, and any mixes or copolymers thereof;
or the group consisting of polyurethane, polyacrylonitrile, and any mixes or copolymers thereof;
or the group consisting of polyurethane, and any mixes or copolymers thereof, and any copolymers of polyethylene;
or the group consisting of polyurethane, and any mixes or copolymers thereof, and any copolymers of polyacrylonitrile;
or the group consisting of polyurethane, and any mixes or copolymers thereof, and poly(acrylonitrile-butadiene) copolymer;
or the group consisting of polyurethane, and any mixes or copolymers thereof, and poly(ethylene-vinyl acetate) copolymer.

In another embodiment, said at least one plastic polymer is selected from the group consisting of synthetic polyamide, polyacrylate, polyvinyl chloride, polyacrylonitrile, and any mixes or copolymers thereof, and any copolymers of polyethylene;
or the group consisting of synthetic polyamide, polyacrylate, polyvinyl chloride, polyacrylonitrile, and any mixes or copolymers thereof, and poly(ethylene-vinyl acetate) copolymer;
or the group consisting of synthetic polyamide, polyacrylate, polyvinyl chloride, and any mixes or copolymers thereof, and any copolymers of polyethylene and copolymers of polyacrylonitrile;
or the group consisting of synthetic polyamide, polyacrylate, polyvinyl chloride, and any mixes or copolymers thereof, poly(acrylonitrile-butadiene) copolymer and any copolymers of polyethylene;
or the group consisting of synthetic polyamide, polyacrylate, polyvinyl chloride, and any mixes or copolymers thereof, poly(ethylene-vinyl acetate) copolymer, and any copolymers of polyacrylonitrile;
or the group consisting of synthetic polyamide, polyacrylate, polyvinyl chloride, and any mixes or copolymers thereof, poly(acrylonitrile-butadiene) copolymer and poly(ethylene-vinyl acetate) copolymer;
or the group consisting of synthetic polyamide, polyacrylate, polyacrylonitrile, and any mixes or copolymers thereof, and any copolymers of polyethylene;
or the group consisting of synthetic polyamide, polyacrylate, polyacrylonitrile, and any mixes or copolymers thereof, and poly(ethylene-vinyl acetate) copolymer;
or the group consisting of synthetic polyamide, polyacrylate, and any mixes or copolymers thereof, and any copolymers of polyethylene and copolymers of polyacrylonitrile;
or the group consisting of synthetic polyamide, polyacrylate, and any mixes or copolymers thereof, poly(acrylonitrile-butadiene) copolymer and any copolymers of polyethylene;
or the group consisting of synthetic polyamide, polyacrylate, and any mixes or copolymers thereof, poly(ethylene-vinyl acetate) copolymer, and any copolymers of polyacrylonitrile;
or the group consisting of synthetic polyamide, polyacrylate, and any mixes or copolymers thereof, poly(acrylonitrile-butadiene) copolymer and poly(ethylene-vinyl acetate) copolymer;
or the group consisting of synthetic polyamide, polyacrylate, polyvinyl chloride, polyacrylonitrile, and any mixes or copolymers thereof;
or the group consisting of synthetic polyamide, polyacrylate, polyvinyl chloride, and any mixes or copolymers thereof, and any copolymers of polyacrylonitrile;
or the group consisting of synthetic polyamide, polyacrylate, polyvinyl chloride, and any mixes or copolymers thereof, poly(acrylonitrile-butadiene) copolymer;
or the group consisting of synthetic polyamide, polyacrylate, polyvinyl chloride, and any mixes or copolymers thereof, and any copolymers of polyethylene;
or the group consisting of synthetic polyamide, polyacrylate, polyvinyl chloride, and any mixes or copolymers thereof, and poly(ethylene-vinyl acetate) copolymer;
or the group consisting of synthetic polyamide, polyacrylate, polyvinyl chloride, and any mixes or copolymers thereof;
or the group consisting of synthetic polyamide, polyacrylate, polyacrylonitrile, and any mixes or copolymers thereof;
or the group consisting of synthetic polyamide, polyacrylate, and any mixes or copolymers thereof, and any copolymers of polyethylene;
or the group consisting of synthetic polyamide, polyacrylate, and any mixes or copolymers thereof, and any copolymers of polyacrylonitrile;
or the group consisting of synthetic polyamide, polyacrylate, and any mixes or copolymers thereof, and poly(acrylonitrile-butadiene) copolymer;
or the group consisting of synthetic polyamide, polyacrylate, and any mixes or copolymers thereof, and poly(ethylene-vinyl acetate) copolymer.

In one embodiment, the polyethylene copolymer is poly(ethylene-vinyl acetate). In one embodiment, the polyacrylonitrile copolymer is poly(acrylonitrile-butadiene).

In one embodiment, said least one plastic polymer is selected from the group consisting of polyvinyl chloride, polyacrylate, synthetic polyamide, polyurethane, polyester, and any mixes or copolymers thereof;
such as the group consisting of polyvinyl chloride, polyacrylate, synthetic polyamide, polyurethane, and any mixes or copolymers thereof;
such as the group consisting of polyvinyl chloride, polyacrylate, synthetic polyamide, and any mixes or copolymers thereof;
such as the group consisting of polyvinyl chloride, polyacrylate, and any mixes or copolymers thereof;
such as the group consisting of polyvinyl chloride, and any mixes or copolymers thereof.

In another embodiment, said at least one plastic polymer is selected from the group consisting of polyvinyl chloride, polyacrylate, synthetic polyamide, polyurethane, polyester, and any mixes or copolymers thereof, and copolymers of polyether-polyurethane, copolymers of polyacrylonitrile and copolymers of polyethylene, and any mixes thereof;
such as the group consisting of polyvinyl chloride, polyacrylate, synthetic polyamide, polyurethane, and any mixes or copolymers thereof, and copolymers of polyether-polyurethane, copolymers of polyacrylonitrile and copolymers of polyethylene, and any mixes thereof;
such as the group consisting of polyvinyl chloride, polyacrylate, synthetic polyamide, and any mixes or copolymers thereof, and copolymers of polyether-polyurethane, copolymers of polyacrylonitrile and polyethylene, and any mixes thereof;
such as the group consisting of polyvinyl chloride, polyacrylate, and any mixes or copolymers thereof, and copolymers of polyether-polyurethane, polyacrylonitrile and copolymers of polyethylene, and any mixes thereof;
such as the group consisting of polyvinyl chloride, and any mixes or copolymers thereof, and copolymers of polyether-polyurethane, copolymers of polyacrylonitrile and copolymers of polyethylene, and any mixes thereof.

In another embodiment, said at least one plastic polymer is selected from the group consisting of polyvinyl chloride, polyacrylate, synthetic polyamide, polyurethane, polyester, and any mixes or copolymers thereof, and copolymers of polyacrylonitrile and copolymers of polyethylene, and any mixes thereof;
such as the group consisting of polyvinyl chloride, polyacrylate, synthetic polyamide, polyurethane, and any mixes or copolymers thereof, and copolymers of polyacrylonitrile and copolymers of polyethylene, and any mixes thereof;
such as the group consisting of polyvinyl chloride, polyacrylate, synthetic polyamide, and any mixes or copolymers thereof, and copolymers of polyacrylonitrile and copolymers of polyethylene, and any mixes thereof;
such as the group consisting of polyvinyl chloride, polyacrylate, and any mixes or copolymers thereof, and copolymers of polyacrylonitrile and copolymers of polyethylene, and any mixes thereof;
such as the group consisting of polyvinyl chloride, and any mixes or copolymers thereof, and copolymers of polyacrylonitrile and copolymers of polyethylene, and any mixes thereof.

In another embodiment, said at least one plastic polymer is selected from the group consisting of polyvinyl chloride, polyacrylate, synthetic polyamide, polyurethane, polyester, and any mixes or copolymers thereof, and copolymers of polyacrylonitrile, and any mixes thereof;
such as the group consisting of polyvinyl chloride, polyacrylate, synthetic polyamide, polyurethane, and any mixes or copolymers thereof, and copolymers of polyacrylonitrile, and any mixes thereof;
such as the group consisting of polyvinyl chloride, polyacrylate, synthetic polyamide, and any mixes or copolymers thereof, and copolymers of polyacrylonitrile, and any mixes thereof;
such as the group consisting of polyvinyl chloride, polyacrylate, and any mixes or copolymers thereof, and copolymers of polyacrylonitrile, and any mixes thereof;
such as the group consisting of polyvinyl chloride, and any mixes or copolymers thereof, and copolymers of polyacrylonitrile, and any mixes thereof.

In another embodiment, said least one plastic polymer is selected from the group consisting of polyvinyl chloride, polyacrylate, synthetic polyamide, polyurethane, polyester, and any mixes or copolymers thereof, and copolymers of polyacrylonitrile and copolymers of polyethylene;
such as the group consisting of polyvinyl chloride, polyacrylate, synthetic polyamide, polyurethane, and any mixes or copolymers thereof, and copolymers of polyacrylonitrile and copolymers of polyethylene;
such as the group consisting of polyvinyl chloride, polyacrylate, synthetic polyamide, and any mixes or copolymers thereof, and copolymers of polyacrylonitrile and copolymers of polyethylene;
such as the group consisting of polyvinyl chloride, polyacrylate, and any mixes or copolymers thereof, and copolymers of polyacrylonitrile and copolymers of polyethylene;
such as the group consisting of polyvinyl chloride, and any mixes or copolymers thereof, and copolymers of polyacrylonitrile and copolymers of polyethylene.

In another embodiment, said at least one plastic polymer is selected from the group consisting of polyvinyl chloride, polyacrylate, synthetic polyamide, polyurethane, polyester, and any mixes or copolymers thereof, and copolymers of polyacrylonitrile;
such as the group consisting of polyvinyl chloride, polyacrylate, synthetic polyamide, polyurethane, and any mixes or copolymers thereof, and copolymers of polyacrylonitrile;
such as the group consisting of polyvinyl chloride, polyacrylate, synthetic polyamide, and any mixes or copolymers thereof, and copolymers of polyacrylonitrile;
such as the group consisting of polyvinyl chloride, polyacrylate, and any mixes or copolymers thereof, and copolymers of polyacrylonitrile;
such as the group consisting of polyvinyl chloride, and any mixes or copolymers thereof, and copolymers of polyacrylonitrile.

In another embodiment, said at least one plastic polymer is selected from the group consisting of polyester, polyethylene terephthalate, polypropylene terephthalate, polycarbonate, polyacrylate, poly(ethylene-vinyl acetate), and any mixes or copolymers thereof.

In another embodiment, said at least one plastic polymer is selected from the group consisting of polyethylene terephthalate, polypropylene terephthalate, polycarbonate, polyacrylate, poly(ethylene-vinyl acetate), and any mixes or copolymers thereof.

In another embodiment, said at least one plastic polymer is selected from the group consisting of polyester, polyethylene terephthalate, polypropylene terephthalate, polycarbonate, polyacrylate, and any mixes or copolymers thereof.

In another embodiment, said at least one plastic polymer is selected from the group consisting of polyethylene terephthalate, polypropylene terephthalate, polycarbonate, polyacrylate, and any mixes or copolymers thereof.

In another embodiment, said at least one plastic polymer is selected from the group consisting of polyester, polyethylene terephthalate, polypropylene terephthalate, and any mixes or copolymers thereof.

In another embodiment, said at least one plastic polymer is selected from the group consisting of polyethylene terephthalate, polypropylene terephthalate, and any mixes or copolymers thereof.

In another embodiment, said at least one plastic polymer is selected from the group consisting of polyether, polyglycol, and any mixes or copolymers thereof.

In another embodiment, said at least one plastic polymer is selected from the group consisting of polyurethane, and any mixes or copolymers thereof.

In another embodiment, said at least one plastic polymer is selected from the group consisting of polycarbonate, and any mixes or copolymers thereof.

In another embodiment, said at least one plastic polymer is selected from the group consisting of synthetic polyamide, and any mixes or copolymers thereof.

In yet another embodiment, said at least one plastic polymer is selected from the group consisting of polyvinyl chloride, polyvinylidene chloride, polyacrylate, polyacrylonitrile, and any mixes or copolymers thereof.

In the building and construction industry, there is a high demand on long-lasting materials or materials that can resist external forces. The materials often contain mixtures of polymers and/or copolymers, such as exemplified but not limited to the ones listed in the following embodiment. Thus, in one embodiment, the polymer is selected from the group consisting of polypropylene, polyurethane, and polyacrylate, and any mixes or copolymers thereof.

In the transportation industry, there is a high demand on materials that can resist external forces. The materials often contain mixtures of polymers and/or copolymers, such as exemplified but not limited to the ones listed in the following embodiment. Thus, in one embodiment, the polymer is selected from the group consisting of polypropylene and polyurethane.

In the packaging industry, it is common to use different plastic polymers for different part of a package. Packaging materials often contain mixtures of polymers and/or copolymers, such as exemplified but not limited to the ones listed in the following embodiment. In one embodiment, the polymer is selected from the group consisting of polypropylene and polyethylene terephthalate, and any mixes or copolymers thereof.

A large number of plastic polymers are used in the clothing industry and clothing materials often contain mixtures of polymers and/or copolymers, such as exemplified but not limited to the ones listed in the following embodiment. Thus, in one embodiment, the polymer is selected from the group consisting of polypropylene, synthetic polyamide, polyacrylate, and polyester and any mixes or copolymers thereof. In one embodiment, the polymer is selected from the group consisting of synthetic polyamide and polyacrylate and any mixes or copolymers thereof. In one embodiment, the polymer is selected from the group consisting of synthetic polyamide, polyester, and polyacrylate. In one embodiment, the polymer is selected from the group consisting of synthetic polyamide and polyester. In one embodiment, the polymer is selected from the group consisting of synthetic polyamide and polyacrylate. In one embodiment, the polymer is selected from the group consisting of polyacrylate and polyester.

It is common to make and use polymer materials comprising more than one polymer, such as for example two polymers, such as for example three polymers, such as for example four polymers, such as for example five polymers, such as for example six polymers. One example is in the textile industry, where it is common with material blends, such as polyester or any form of cellulose, blended with for example copolymer comprising polyether and polyurethane. Material blends may include mixes of any fibers or yarns comprising different plastic polymers together with natural fibers. Such natural fibers include, for example, sheep wool, camel hair, cotton and cashmere. Material blends, mixtures and combinations are often produced in the textile industry for functional and aesthetic advantages and/or cost efficiency. Thus, degradation of materials that comprise copolymers, different mixes of synthetic and natural polymers, and any mixes thereof, is needed for handling waste produced by the textile industry. It is envisioned that complete clothing items, whole or processed into fragments could be digested without the need to separate different polymers from each other. As such, in embodiments, the material comprises at least one further polymer, such as one further polymer, such as two further polymers, such as three further polymers, such as four further polymers, such as five further polymers, such as six further polymers.

In one embodiment, the polymer is a copolymer comprising polyether and polyurethane, optionally said copolymer comprises a further polymer selected from the list consisting of polyamide, cellulose, and polyester. In a related embodiment, said further polymer is selected from the list consisting of polyamide and cellulose. In a related embodiment, said further polymer is selected from the list consisting of polyamide and polyester. In a related embodiment, said further polymer is selected from the list consisting of polyester and cellulose.

In one embodiment, the polymer is a synthetic polymer. As used herein, a synthetic polyamide is made up of repeating units of the same kind. As such, is different from for example a protein, which consists of different unit and is obtained in a step-by-step type polymerization. A synthetic polymer may for example be obtained by step-growth polymerization. Examples of synthetic polymers are for example nylons. In one embodiment, the synthetic polymer is a synthetic polyamide is selected from the group consisting of PA 6; PA 6,6; PA 10; and PA 12. In one embodiment, the synthetic polymer is a polyester is selected from the group consisting of polyethylene terephthalate, polypropylene terephthalate, polycaprolactone, and polyethylene adipate.

In one embodiment, said plastic polymer is a copolymer selected from the group consisting of polyether copolymers, polyurethane copolymers, polyacrylonitrile copolymers, and polyethylene copolymers, and any mixes thereof;
or the group consisting of polyether copolymers, polyurethane copolymers, and polyacrylonitrile copolymers, and any mixes thereof;
the group consisting of polyether copolymers, polyurethane copolymers, and polyethylene copolymers, and any mixes thereof;
or the group consisting of polyether copolymers, and polyurethane copolymers, and any mixes thereof;
or the group consisting of polyacrylonitrile copolymers, and polyethylene copolymers, and any mixes thereof.

In one embodiment, said copolymer is a polyether-polyurethane copolymer. In another embodiment, said copolymer is a polyacrylonitrile copolymer, such as poly(acrylonitrile-butadiene). In another embodiment, said copolymer is a polyethylene copolymer, such as poly(ethylene-vinyl acetate).

The material comprising at least one plastic polymer as discussed herein may be a copolymer and may further comprise an additional at least one polymer. Thus in some embodiments, said material comprising any of said copolymers, optionally comprises a further polymer. Said further polymer may be synthetic or natural polymer. In some embodiments, said further polymer is selected from the group consisting of polyamide, cellulose, and polyester.

In one embodiment, the polymer is a copolymer selected from the group consisting of polyether copolymers, polyurethane copolymers, polyacrylonitrile copolymers, and polyethylene copolymers, and any mixes thereof, optionally said copolymer comprises a further polymer. In some embodiments, said further polymer is selected from the list consisting of polyamide, cellulose, and polyester.

A polyether copolymer may be a polyether-polyurethane copolymer. A polyurethane copolymer may be a polyether-polyurethane copolymer. A polyethylene copolymer may be poly(ethylene-vinyl acetate). A polyacrylonitrile copolymer may be poly(acrylonitrile-butadiene). Polyacrylonitrile copolymers are also called nitriles. Polyacrylonitrile copolymers are coplymers of acrylonitriles and various other monomers. Such monomers may be styrene, butadiene and methyl acetate. Poly(acrylonitrile-butadiene) rubbers are important elastomers.

G. mellonella, A. diaperinus and T. molitor are known to survive in a variety of conditions, however it is envisioned that conditions may be adapted to provide for efficient digestion of plastic polymers. For example, the life cycle of G. mellonella comprises a larval stage that transforms within 6-8 weeks into pupae and finally into moth. The larvae are fairly large in size, the last instar larva is 2 cm long and 250 mg, or even up to 400-500 mg, in weight and can be reared at various temperatures (20-30° C.).

In one embodiment, the method is performed at a temperature of approximately from 4° C. to 40° C., such as approximately from 15° C. to 35° C., preferably of approximately from 20° C. to 30° C., most preferably of approximately from 25° C. to 27° C.

In one embodiment, the method is performed at a humidity of from 30% to 99%. In one preferred embodiment, the humidity is maintained at approximately 80.0+/−10%, such as approximately 80.0+/−5%.

In one particular embodiment, the method comprises placing the larva in the container at a density of 0.2-5 larvae per cm², such as 0.2-3 larvae per cm², such as 0.2-2 larvae per cm² or such as 0.5-3 larvae per cm², such as 0.5-2 larvae per cm², such as 1-2 larvae per cm². As apparent for a person of skill in the art, higher density may be preferable for industrial purposes in order to provide higher production of biomass per area unit. However, high density is often avoided as it can contribute to cannibalism of the larvae. In one embodiment, the population of larvae exhibit no significant cannibalism, such as no cannibalism at all or less than 5%, such as less than 3%, such as less than 2%, such as less than 1% cannibalism. The skilled person will appreciate that a cannibal is defined as an individual that consumes other individual larvae of the same species as at least part of their diet. The percentage of cannibalism may be based on the population and may be calculated as the number of other individual larvae that are at least partly consumed over the total number of larvae in the population.

Without being bound by any theory, it is considered that starvation of larva prior to providing the larva will plastic polymer based feed may lead to more efficient consumption, and thus degradation, of plastic polymers as disclosed herein. In one embodiment, the larva is subjected to starvation prior to adding any feed, such as said at least one plastic polymer. For example, the starvation may prolong for approximately 1 to 20 days, such as approximately for 1 to 10, such as approximately for 1 to 5 days, such as approximately for 2 days.

The skilled person will appreciate that any group of polymers listed in the context of the first aspect as disclosed herein is equally relevant for the second and fourth aspects as described below and is not repeated here only for the sake of brevity.

In a second aspect, there is provided use of at least one larva selected from the group consisting of G. mellonella, A. diaperinus, and T. molitor for digestion of a plastic-comprising material. The material comprises at least one plastic polymer selected from the group consisting of polyethylene terephthalate, polypropylene, polypropylene terephthalate, polyether, polyurethane, polyester, polyacrylate, polyglycol, polyvinyl chloride, polycarbonate, polyvinylidene chloride, synthetic polyamide, and any mixes or copolymers thereof.

In one embodiment, the polymer is selected from the group consisting of polypropylene, polyurethane, synthetic polyamide, polyester, polyacrylate, and any mixes or copolymers thereof.

In one embodiment, the polymer is selected from the group consisting of polypropylene, polyurethane, synthetic polyamide, polyester, and any mixes or copolymers thereof.

In one embodiment, the polymer is selected from the group consisting of polypropylene, polyurethane, and polyacrylate, and any mixes or copolymers thereof.

In one embodiment, the polymer is selected from the group consisting of polypropylene and polyurethane.

In one embodiment, the polymer is selected from the group consisting of synthetic polyamide, polyester, and polyacrylate.

In one embodiment, the polymer is selected from the group consisting of a copolymer comprising polyether and polyurethane, optionally comprising a further polymer selected from the list consisting of a synthetic polyamide, cellulose and polyester.

In one embodiment, the polyester is selected from the group consisting of polyethylene terephthalate, polypropylene terephthalate, polycaprolactone, and polyethylene adipate.

In one embodiment, the polyamide is selected from the group consisting of PA 6; PA 6,6; PA 10; and PA 12.

In a third aspect, there is provided a product obtained by the method according to any one of the first aspect, the second aspect, and the fourth aspect as described herein. In one embodiment, the product comprises biomass. In another embodiment, the product consists of biomass. The biomass produced by the methods disclosed herein is useful for many purposes, of which some are mentioned below. In one embodiment, the biomass comprises pupae of G. mellonella, and/or larvae of G. mellonella, and/or feces of said larvae, which may be extracted from the biomass. In one embodiment, the biomass comprises pupae of G. mellonella. Pupae may be used to maintain a population of larvae that can produce biomass by degradation of at least one plastic polymer. In one embodiment, the biomass comprises larvae of G. mellonella. The larvae may comprise nutrients in the form of lipids and/or protein. In one embodiment, the biomass comprises feces of said larvae of G. mellonella. Feces of the larvae may serve useful as fertilizer. In one embodiment, the biomass further comprises polyethylene glycol. In one embodiment, the biomass comprises pupae of T. molitor, and/or larvae of T. molitor, and/or feces of said larvae, which may be extracted from the biomass. In one embodiment, the biomass comprises pupae of T. molitor. Pupae may be used to maintain a population of larvae that can produce biomass by degradation of at least one plastic polymer. In one embodiment, the biomass comprises larvae of T. molitor. The larvae may comprise nutrients in the form of lipids and/or protein. In one embodiment, the biomass comprises feces of said larvae of T. molitor. Feces of the larvae may serve useful as fertilizer. In one embodiment, the biomass further comprises polyethylene glycol.

In one embodiment, the biomass comprises pupae of A. diaperinus, and/or larvae of A. diaperinus, and/or feces of said larvae, which may be extracted from the biomass. In one embodiment, the biomass comprises pupae of A. diaperinus. Pupae may be used to maintain a population of larvae that can produce biomass by degradation of at least one plastic polymer. In one embodiment, the biomass comprises larvae of A. diaperinus. The larvae may comprise nutrients in the form of lipids and/or protein. In one embodiment, the biomass comprises feces of said larvae of A. diaperinus. Feces of the larvae may serve useful as fertilizer. In one embodiment, the biomass further comprises polyethylene glycol.

Thus, in one embodiment there is provided a fertilizer comprising biomass obtained by a method for degrading one or more plastic-comprising material as disclosed herein. In one embodiment, said fertilizer comprises biomass in the form of feces. In another embodiment, there is provide a animal feed comprising biomass obtained by a method for degrading one or more plastic-comprising material as disclosed herein. In one embodiment, said animal feed comprises biomass in the form of larvae.

In a related fourth aspect, there is provide a method for producing biomass, comprising the steps of
contacting at least one larva of the species G. mellonella and/or at least one larva of the species T. molitor with at least one or more plastic—comprising materials under conditions permissive for said at least one larva to digest said material, wherein said material comprises at least one plastic polymer selected from the group consisting of polyethylene terephthalate, polypropylene, polyurethane, polyester, polyacrylate, polyglycol, polyvinyl chloride, polycarbonate, polyvinylidene chloride, synthetic polyamide, polypropylene terephthalate, polyether, and any mixes or copolymers thereof,
wherein the digestion of said material leads to production of biomass and
harvesting the biomass.

As stated above, the skilled person will appreciate that any group of polymers listed herein is equally applicable to the first, second and fourth aspects as described herein and is not repeated in the connection with each aspect merely for the sake of brevity. Thus the following groups of polymers listed below, are equally relevant for the first, second and fourth aspects, wherein the polymer may be selected from any one of the disclosed groups.

Thus, in one embodiment, said polymer is selected from the group consisting of polypropylene. In one embodiment, said polymer is selected from the group consisting of polyurethane. In one embodiment, said polymer is selected from the group consisting of synthetic polyamide. In one embodiment, said polymer is selected from the group consisting of polyacrylate. In one embodiment, said polymer is selected from the group consisting of polyethylene terephthalate. In one embodiment, said polymer is selected from the group consisting of polyester. In one embodiment, said polymer is selected from the group consisting of polypropylene, and polyurethane. In one embodiment, said polymer is selected from the group consisting of polypropylene, and synthetic polyamide. In one embodiment, said polymer is selected from the group consisting of polypropylene, and polyacrylate. In one embodiment, said polymer is selected from the group consisting of polypropylene, and polyethylene terephthalate. In one embodiment, said polymer is selected from the group consisting of polypropylene, and polyester. In one embodiment, said polymer is selected from the group consisting of polyurethane, and synthetic polyamide. In one embodiment, said polymer is selected from the group consisting of polyurethane, and polyacrylate. In one embodiment, said polymer is selected from the group consisting of polyurethane, and polyethylene terephthalate. In one embodiment, said polymer is selected from the group consisting of polyurethane, and polyester. In one embodiment, said polymer is selected from the group consisting of synthetic polyamide, and polyacrylate. In one embodiment, said polymer is selected from the group consisting of synthetic polyamide, and polyethylene terephthalate. In one embodiment, said polymer is selected from the group consisting of synthetic polyamide, and polyester. In one embodiment, said polymer is selected from the group consisting of polyacrylate, and polyethylene terephthalate. In one embodiment, said polymer is selected from the group consisting of polyacrylate, and polyester. In one embodiment, said polymer is selected from the group consisting of polyethylene terephthalate, and polyester. In one embodiment, said polymer is selected from the group consisting of polypropylene, polyurethane, and synthetic polyamide. In one embodiment, said polymer is selected from the group consisting of polypropylene, polyurethane, and polyacrylate. In one embodiment, said polymer is selected from the group consisting of polypropylene, polyurethane, and polyethylene terephthalate. In one embodiment, said polymer is selected from the group consisting of polypropylene, polyurethane, and polyester. In one embodiment, said polymer is selected from the group consisting of polypropylene, synthetic polyamide, and polyacrylate. In one embodiment, said polymer is selected from the group consisting of polypropylene, synthetic polyamide, and polyethylene terephthalate. In one embodiment, said polymer is selected from the group consisting of polypropylene, synthetic polyamide, and polyester. In one embodiment, said polymer is selected from the group consisting of polypropylene, polyacrylate, and polyethylene terephthalate. In one embodiment, said polymer is selected from the group consisting of polypropylene, polyacrylate, and polyester. In one embodiment, said polymer is selected from the group consisting of polypropylene, polyethylene terephthalate, and polyester. In one embodiment, said polymer is selected from the group consisting of polyurethane, synthetic polyamide, and polyacrylate. In one embodiment, said polymer is selected from the group consisting of polyurethane, synthetic polyamide, and polyethylene terephthalate. In one embodiment, said polymer is selected from the group consisting of polyurethane, synthetic polyamide, and polyester. In one embodiment, said polymer is selected from the group consisting of polyurethane, polyacrylate, and polyethylene terephthalate. In one embodiment, said polymer is selected from the group consisting of polyurethane, polyacrylate, and polyester. In one embodiment, said polymer is selected from the group consisting of polyurethane, polyethylene terephthalate, and polyester. In one embodiment, said polymer is selected from the group consisting of synthetic polyamide, polyacrylate, and polyethylene terephthalate. In one embodiment, said polymer is selected from the group consisting of synthetic polyamide, polyacrylate, and polyester. In one embodiment, said polymer is selected from the group consisting of synthetic polyamide, polyethylene terephthalate, and polyester. In one embodiment, said polymer is selected from the group consisting of polyacrylate, polyethylene terephthalate, and polyester. In one embodiment, said polymer is selected from the group consisting of polypropylene, polyurethane, synthetic polyamide, and polyacrylate. In one embodiment, said polymer is selected from the group consisting of polypropylene, polyurethane, synthetic polyamide, and polyethylene terephthalate. In one embodiment, said polymer is selected from the group consisting of polypropylene, polyurethane, synthetic polyamide, and polyester. In one embodiment, said polymer is selected from the group consisting of polypropylene, polyurethane, polyacrylate, and polyethylene terephthalate. In one embodiment, said polymer is selected from the group consisting of polypropylene, polyurethane, polyacrylate, and polyester. In one embodiment, said polymer is selected from the group consisting of polypropylene, polyurethane, polyethylene terephthalate, and polyester. In one embodiment, said polymer is selected from the group consisting of polypropylene, synthetic polyamide, polyacrylate, and polyethylene terephthalate. In one embodiment, said polymer is selected from the group consisting of polypropylene, synthetic polyamide, polyacrylate, and polyester. In one embodiment, said polymer is selected from the group consisting of polypropylene, synthetic polyamide, polyethylene terephthalate, and polyester. In one embodiment, said polymer is selected from the group consisting of polypropylene, polyacrylate, polyethylene terephthalate, and polyester. In one embodiment, said polymer is selected from the group consisting of polyurethane, synthetic polyamide, polyacrylate, and polyethylene terephthalate. In one embodiment, said polymer is selected from the group consisting of polyurethane, synthetic polyamide, polyacrylate, and polyester. In one embodiment, said polymer is selected from the group consisting of polyurethane, synthetic polyamide, polyethylene terephthalate, and polyester. In one embodiment, said polymer is selected from the group consisting of polyurethane, polyacrylate, polyethylene terephthalate, and polyester. In one embodiment, said polymer is selected from the group consisting of synthetic polyamide, polyacrylate, polyethylene terephthalate, and polyester. In one embodiment, said polymer is selected from the group consisting of polypropylene, polyurethane, synthetic polyamide, polyacrylate, and polyethylene terephthalate. In one embodiment, said polymer is selected from the group consisting of polypropylene, polyurethane, synthetic polyamide, polyacrylate, and polyester. In one embodiment, said polymer is selected from the group consisting of polypropylene, polyurethane, synthetic polyamide, polyethylene terephthalate, and polyester. In one embodiment, said polymer is selected from the group consisting of polypropylene, polyurethane, polyacrylate, polyethylene terephthalate, and polyester. In one embodiment, said polymer is selected from the group consisting of polypropylene, synthetic polyamide, polyacrylate, polyethylene terephthalate, and polyester. In one embodiment, said polymer is selected from the group consisting of polyurethane, synthetic polyamide, polyacrylate, polyethylene terephthalate, and polyester. In one embodiment, said polymer is selected from the group consisting of polypropylene, polyurethane, synthetic polyamide, polyacrylate, polyethylene terephthalate, and polyester.

In one embodiment, said polymer is selected from the group consisting of polyamide, polyester, polyacrylate, polypropylene, polyamide/polyether-polyurethane copolymer, cotton/polyester/polyether-polyurethane copolymer, polyurethane; or the group consisting of polyamide, polyacrylate, polypropylene, polyamide/polyether-polyurethane copolymer, cotton/polyester/polyether-polyurethane copolymer, polyurethane; or the group consisting of polyamide, polyester, polypropylene, polyamide/polyether-polyurethane copolymer, cotton/polyester/polyether-polyurethane copolymer, polyurethane; or the group consisting of polyamide, polyester, polyacrylate, polyamide/polyether-polyurethane copolymer, cotton/polyester/polyether-polyurethane copolymer, polyurethane; or the group consisting of polyamide, polyester, polyacrylate, polypropylene, cotton/polyester/polyether-polyurethane copolymer, polyurethane; or the group consisting of polyamide, polyester, polyacrylate, polypropylene, polyamide/polyether-polyurethane copolymer, polyurethane; or the group consisting of polyamide, polyester, polyacrylate, polypropylene, polyamide/polyether-polyurethane copolymer, cotton/polyester/polyether-polyurethane copolymer; or the group consisting of polyacrylate, polypropylene, polyamide/polyether-polyurethane copolymer, cotton/polyester/polyether-polyurethane copolymer, polyurethane; or the group consisting of polyacrylate, polypropylene, polyamide/polyether-polyurethane copolymer, cotton/polyester/polyether-polyurethane copolymer, polyurethane; or
the group consisting of polyester, polypropylene, polyamide/polyether-polyurethane copolymer, cotton/polyester/polyether-polyurethane copolymer, polyurethane; or the group consisting of polyester, polyacrylate, polyamide/polyether-polyurethane copolymer, cotton/polyester/polyether-polyurethane copolymer, polyurethane; or the group consisting of polyester, polyacrylate, polypropylene, cotton/polyester/polyether-polyurethane copolymer, polyurethane; or the group consisting of polyester, polyacrylate, polypropylene, polyamide/polyether-polyurethane copolymer, polyurethane; or the group consisting of polyester, polyacrylate, polypropylene, polyamide/polyether-polyurethane copolymer, cotton/polyester/polyether-polyurethane copolymer; or the group consisting of polyamide, polypropylene, polyamide/polyether-polyurethane copolymer, cotton/polyester/polyether-polyurethane copolymer, polyurethane; or the group consisting of polyamide, polyacrylate, polyamide/polyether-polyurethane copolymer, cotton/polyester/polyether-polyurethane copolymer, polyurethane; or the group consisting of polyamide, polyacrylate, polypropylene, cotton/polyester/polyether-polyurethane copolymer, polyurethane; or the group consisting of polyamide, polyacrylate, polypropylene, polyamide/polyether-polyurethane copolymer, polyurethane; or the group consisting of polyamide, polyacrylate, polypropylene, polyamide/polyether-polyurethane copolymer, cotton/polyester/polyether-polyurethane copolymer; or the group consisting of polyamide, polyester, polyamide/polyether-polyurethane copolymer, cotton/polyester/polyether-polyurethane copolymer, polyurethane; or the group consisting of polyamide, polyester, polypropylene, cotton/polyester/polyether-polyurethane copolymer, polyurethane; or the group consisting of polyamide, polyester, polypropylene, polyamide/polyether-polyurethane copolymer, polyurethane; or the group consisting of polyamide, polyester, polypropylene, polyamide/polyether-polyurethane copolymer, cotton/polyester/polyether-polyurethane copolymer; or the group consisting of polyamide, polyester, polyacrylate, cotton/polyester/polyether-polyurethane copolymer, polyurethane; or the group consisting of polyamide, polyester, polyacrylate, polyamide/polyether-polyurethane copolymer, polyurethane; or the group consisting of polyamide, polyester, polyacrylate, polyamide/polyether-polyurethane copolymer, cotton/polyester/polyether-polyurethane copolymer; or the group consisting of polyamide, polyester, polyacrylate, polypropylene, polyurethane; or the group consisting of polyamide, polyester, polyacrylate, polypropylene, cotton/polyester/polyether-polyurethane copolymer; or the group consisting of polyamide, polyester, polyacrylate, polypropylene, polyamide/polyether-polyurethane copolymer.

In one embodiment, said polymer is selected from the group consisting of polyamide, polyester, polyacrylate, polypropylene, polyamide/polyether-polyurethane copolymer and cotton/polyester/polyether-polyurethane copolymer; or the group consisting of polyamide, polyacrylate, polypropylene, polyamide/polyether-polyurethane copolymer and cotton/polyester/polyether-polyurethane copolymer; or the group consisting of polyamide, polyester, polypropylene, polyamide/polyether-polyurethane copolymer and cotton/polyester/polyether-polyurethane copolymer; or the group consisting of polyamide, polyester, polyacrylate, polyamide/polyether-polyurethane copolymer and cotton/polyester/polyether-polyurethane copolymer; or the group consisting of polyamide, polyester, polyacrylate, polypropylene and cotton/polyester/polyether-polyurethane copolymer; or the group consisting of polyamide, polyester, polyacrylate, polypropylene and polyamide/polyether-polyurethane copolymer; or the group consisting of polyacrylate, polypropylene, polyamide/polyether-polyurethane copolymer and cotton/polyester/polyether-polyurethane copolymer; or the group consisting of polyester, polypropylene, polyamide/polyether-polyurethane copolymer and cotton/polyester/polyether-polyurethane copolymer; or the group consisting of polyester, polyacrylate, polyamide/polyether-polyurethane copolymer and cotton/polyester/polyether-polyurethane copolymer; or the group consisting of polyester, polyacrylate, polypropylene and cotton/polyester/polyether-polyurethane copolymer; or the group consisting of polyester, polyacrylate, polypropylene and polyamide/polyether-polyurethane copolymer; or the group consisting of polyamide, polypropylene, polyamide/polyether-polyurethane copolymer and cotton/polyester/polyether-polyurethane copolymer; or the group consisting of polyamide, polyacrylate, polyamide/polyether-polyurethane copolymer, cotton/polyester/polyether-polyurethane copolymer; or the group consisting of polyamide, polyacrylate, polypropylene and cotton/polyester/polyether-polyurethane copolymer; or the group consisting of polyamide, polyacrylate, polypropylene and polyamide/polyether-polyurethane copolymer; or the group consisting of polyamide, polyester, polyamide/polyether-polyurethane copolymer and cotton/polyester/polyether-polyurethane copolymer; or the group consisting of polyamide, polyester, polypropylene and cotton/polyester/polyether-polyurethane copolymer; or the group consisting of polyamide, polyester, polypropylene and polyamide/polyether-polyurethane copolymer; or the group consisting of polyamide, polyester, polyacrylate and cotton/polyester/polyether-polyurethane copolymer; or the group consisting of polyamide, polyester, polyacrylate, polypropylene and polyamide/polyether-polyurethane copolymer; or the group consisting of polyamide, polyester, polyacrylate and polypropylene.

In another embodiment, said polymer is selected from the group consisting of polypropylene, polyamide/polyether-polyurethane copolymer, cotton/polyester/polyether-polyurethane copolymer; or such as the group consisting of polyacrylate, polyamide/polyether-polyurethane copolymer, cotton/polyester/polyether-polyurethane copolymer; or such as the group consisting of polyacrylate, polypropylene, cotton/polyester/polyether-polyurethane copolymer; or such as the group consisting of polyacrylate, polypropylene, polyamide/polyether-polyurethane copolymer; or such as the group consisting of polyester, polyamide/polyether-polyurethane copolymer, cotton/polyester/polyether-polyurethane copolymer; or such as the group consisting of polyester, polypropylene, cotton/polyester/polyether-polyurethane copolymer; or such as the group consisting of polyester, polypropylene, polyamide/polyether-polyurethane copolymer; or such as the group consisting of polyacrylate, polyamide/polyether-polyurethane copolymer, cotton/polyester/polyether-polyurethane copolymer; or such as the group consisting of polyester, polyacrylate, cotton/polyester/polyether-polyurethane copolymer; or such as the group consisting of polyester, polyacrylate, polyamide/polyether-polyurethane copolymer; or such as the group consisting of polyacrylate, polypropylene, cotton/polyester/polyether-polyurethane copolymer; or such as the group consisting of polyester, polyacrylate, polypropylene; or such as the group consisting of polyacrylate, polypropylene, polyamide/polyether-polyurethane copolymer; or such as the group consisting of polyamide, polyamide/polyether-polyurethane copolymer, cotton/polyester/polyether-polyurethane copolymer; or such as the group consisting of polyamide, polypropylene, cotton/polyester/polyether-polyurethane copolymer; or such as the group consisting of polyamide, polypropylene, polyamide/polyether-polyurethane copolymer; or such as the group consisting of polyamide, polyacrylate, cotton/polyester/polyether-polyurethane copolymer; or such as the group consisting of polyamide, polyacrylate, polyamide/polyether-polyurethane copolymer; or such as the group consisting of polyamide, polyacrylate, polypropylene; or such as the group consisting of polyester, polyamide/polyether-polyurethane copolymer, cotton/polyester/polyether-polyurethane copolymer; or such as the group consisting of polyamide, 2polyester, cotton/polyester/polyether-polyurethane copolymer; or such as the group consisting of polyamide, polyester, polyamide/polyether-polyurethane copolymer; or such as the group consisting of polyester, polypropylene, cotton/polyester/polyether-polyurethane copolymer; or such as the group consisting of polyamide, polyester, polypropylene; or such as the group consisting of polyester, polypropylene, polyamide/polyether-polyurethane copolymer; or such as the group consisting of polyester, polyacrylate, cotton/polyester/polyether-polyurethane copolymer; or such as the group consisting of polyamide, polyester, polyacryl; or such as the group consisting of polyester, polyacrylate, polyamide/polyether-polyurethane copolymer; or
such as the group consisting of polyester, polyacrylate, polypropylene; or
such as the group consisting of polyamide, polyester, polyacrylate, polypropylene.

In one embodiment, said polymer is selected from the group consisting of polyamid and polyester; or the group consisting of polyamid and polyacryl; or the group consisting of polyamid and polypropylene; or the group consisting of polyamid and polyamide/polyether-polyurethane copolymer; or the group consisting of polyamid and polyamide/polyether-polyurethane copolymer; or the group consisting of polyamid and polyurethane; or the group consisting of polyacrylate and polypropylene; or the group consisting of polyacrylate and polyamide/polyether-polyurethane copolymer; or the group consisting of polyacrylate and polyamide/polyether-polyurethane copolymer; or the group consisting of polyacrylate and polyurethane; or the group consisting of polypropylene and polyamide/polyether-polyurethane copolymer; or the group consisting of polypropylene and polyamide/polyether-polyurethane copolymer; or the group consisting of polypropylene and polyurethane; or the group consisting of polyamide/polyether-polyurethane copolymer and polyamide/polyether-polyurethane copolymer; or the group consisting of polyamide/polyether-polyurethane copolymer and polyurethane; or the group consisting of polyurethane and polyamide/polyether-polyurethane copolymer. In one embodiment, said polymer is polyamide, polyester, polyacrylate, polypropylene, polyamide/polyether-polyurethane copolymer or polyamide/polyether-polyurethane copolymer or polyurethane.

In one embodiment, said polymer is selected from the group consisting of poly(ethylene-vinyl acetate). In one embodiment, said polymer is selected from the group consisting of poly(ethylene-vinyl acetate), and polyurethane. In one embodiment, said polymer is selected from the group consisting of poly(ethylene-vinyl acetate), and synthetic polyamide. In one embodiment, said polymer is selected from the group consisting of poly(ethylene-vinyl acetate), and polyacrylate. In one embodiment, said polymer is selected from the group consisting of poly(ethylene-vinyl acetate), and polyethylene terephthalate. In one embodiment, said polymer is selected from the group consisting of poly(ethylene-vinyl acetate), and polyester. In one embodiment, said polymer is selected from the group consisting of poly(ethylene-vinyl acetate), polyurethane, and synthetic polyamide. In one embodiment, said polymer is selected from the group consisting of poly(ethylene-vinyl acetate), polyurethane, and polyacrylate. In one embodiment, said polymer is selected from the group consisting of poly(ethylene-vinyl acetate), polyurethane, and polyethylene terephthalate. In one embodiment, said polymer is selected from the group consisting of poly(ethylene-vinyl acetate), polyurethane, and polyester. In one embodiment, said polymer is selected from the group consisting of poly(ethylene-vinyl acetate), synthetic polyamide, and polyacrylate. In one embodiment, said polymer is selected from the group consisting of poly(ethylene-vinyl acetate), synthetic polyamide, and polyethylene terephthalate. In one embodiment, said polymer is selected from the group consisting of poly(ethylene-vinyl acetate), synthetic polyamide, and polyester. In one embodiment, said polymer is selected from the group consisting of poly(ethylene-vinyl acetate), polyacrylate, and polyethylene terephthalate. In one embodiment, said polymer is selected from the group consisting of poly(ethylene-vinyl acetate), polyacrylate, and polyester. In one embodiment, said polymer is selected from the group consisting of poly(ethylene-vinyl acetate), polyethylene terephthalate, and polyester. In one embodiment, said polymer is selected from the group consisting of poly(ethylene-vinyl acetate), polyurethane, synthetic polyamide, and polyacrylate. In one embodiment, said polymer is selected from the group consisting of poly(ethylene-vinyl acetate), polyurethane, synthetic polyamide, and polyethylene terephthalate. In one embodiment, said polymer is selected from the group consisting of poly(ethylene-vinyl acetate), polyurethane, synthetic polyamide, and polyester. In one embodiment, said polymer is selected from the group consisting of poly(ethylene-vinyl acetate), polyurethane, polyacrylate, and polyethylene terephthalate. In one embodiment, said polymer is selected from the group consisting of poly(ethylene-vinyl acetate), polyurethane, polyacrylate, and polyester. In one embodiment, said polymer is selected from the group consisting of poly(ethylene-vinyl acetate), polyurethane, polyethylene terephthalate, and polyester. In one embodiment, said polymer is selected from the group consisting of poly(ethylene-vinyl acetate), synthetic polyamide, polyacrylate, and polyethylene terephthalate. In one embodiment, said polymer is selected from the group consisting of poly(ethylene-vinyl acetate), synthetic polyamide, polyacrylate, and polyester. In one embodiment, said polymer is selected from the group consisting of poly(ethylene-vinyl acetate), synthetic polyamide, polyethylene terephthalate, and polyester. In one embodiment, said polymer is selected from the group consisting of poly(ethylene-vinyl acetate), polyacrylate, polyethylene terephthalate, and polyester. In one embodiment, said polymer is selected from the group consisting of poly(ethylene-vinyl acetate), polyurethane, synthetic polyamide, polyacrylate, and polyethylene terephthalate. In one embodiment, said polymer is selected from the group consisting of poly(ethylene-vinyl acetate), polyurethane, synthetic polyamide, polyacrylate, and polyester. In one embodiment, said polymer is selected from the group consisting of poly(ethylene-vinyl acetate), polyurethane, synthetic polyamide, polyethylene terephthalate, and polyester. In one embodiment, said polymer is selected from the group consisting of poly(ethylene-vinyl acetate), polyurethane, polyacrylate, polyethylene terephthalate, and polyester. In one embodiment, said polymer is selected from the group consisting of poly(ethylene-vinyl acetate), synthetic polyamide, polyacrylate, polyethylene terephthalate, and polyester. In one embodiment, said polymer is selected from the group consisting of poly(ethylene-vinyl acetate), polyurethane, synthetic polyamide, polyacrylate, polyethylene terephthalate, and polyester.

In one embodiment, said polymer is selected from the group consisting of poly(acrylonitrile-butadiene) and poly(ethylene-vinyl acetate). In one embodiment, said polymer is selected from the group consisting of poly(acrylonitrile-butadiene) and polyurethane. In one embodiment, said polymer is selected from the group consisting of poly(acrylonitrile-butadiene) and synthetic polyamide. In one embodiment, said polymer is selected from the group consisting of poly(acrylonitrile-butadiene) and polyacrylate. In one embodiment, said polymer is selected from the group consisting of poly(acrylonitrile-butadiene) and polyethylene terephthalate. In one embodiment, said polymer is selected from the group consisting of poly(acrylonitrile-butadiene) and polyester. In one embodiment, said polymer is selected from the group consisting of poly(acrylonitrile-butadiene), poly(ethylene-vinyl acetate), and polyurethane. In one embodiment, said polymer is selected from the group consisting of poly(acrylonitrile-butadiene), poly(ethylene-vinyl acetate), and synthetic polyamide. In one embodiment, said polymer is selected from the group consisting of poly(acrylonitrile-butadiene), poly(ethylene-vinyl acetate), and polyacrylate. In one embodiment, said polymer is selected from the group consisting of poly(acrylonitrile-butadiene), poly(ethylene-vinyl acetate), and polyethylene terephthalate. In one embodiment, said polymer is selected from the group consisting of poly(acrylonitrile-butadiene), poly(ethylene-vinyl acetate), and polyester. In one embodiment, said polymer is selected from the group consisting of poly(acrylonitrile-butadiene), polyurethane, and synthetic polyamide. In one embodiment, said polymer is selected from the group consisting of poly(acrylonitrile-butadiene), polyurethane, and polyacrylate. In one embodiment, said polymer is selected from the group consisting of poly(acrylonitrile-butadiene), polyurethane, and polyethylene terephthalate. In one embodiment, said polymer is selected from the group consisting of poly(acrylonitrile-butadiene), polyurethane, and polyester. In one embodiment, said polymer is selected from the group consisting of poly(acrylonitrile-butadiene), synthetic polyamide, and polyacrylate. In one embodiment, said polymer is selected from the group consisting of poly(acrylonitrile-butadiene), synthetic polyamide, and polyethylene terephthalate. In one embodiment, said polymer is selected from the group consisting of poly(acrylonitrile-butadiene), synthetic polyamide, and polyester. In one embodiment, said polymer is selected from the group consisting of poly(acrylonitrile-butadiene), polyacrylate, and polyethylene terephthalate. In one embodiment, said polymer is selected from the group consisting of poly(acrylonitrile-butadiene), polyacrylate, and polyester. In one embodiment, said polymer is selected from the group consisting of poly(acrylonitrile-butadiene), polyethylene terephthalate, and polyester. In one embodiment, said polymer is selected from the group consisting of poly(acrylonitrile-butadiene), poly(ethylene-vinyl acetate), polyurethane, and synthetic polyamide. In one embodiment, said polymer is selected from the group consisting of poly(acrylonitrile-butadiene), poly(ethylene-vinyl acetate), polyurethane, and polyacrylate. In one embodiment, said polymer is selected from the group consisting of poly(acrylonitrile-butadiene), poly(ethylene-vinyl acetate), polyurethane, and polyethylene terephthalate. In one embodiment, said polymer is selected from the group consisting of poly(acrylonitrile-butadiene), poly(ethylene-vinyl acetate), polyurethane, and polyester. In one embodiment, said polymer is selected from the group consisting of poly(acrylonitrile-butadiene), poly(ethylene-vinyl acetate), synthetic polyamide, and polyacrylate. In one embodiment, said polymer is selected from the group consisting of poly(acrylonitrile-butadiene), poly(ethylene-vinyl acetate), synthetic polyamide, and polyethylene terephthalate. In one embodiment, said polymer is selected from the group consisting of poly(acrylonitrile-butadiene), poly(ethylene-vinyl acetate), synthetic polyamide, and polyester. In one embodiment, said polymer is selected from the group consisting of poly(acrylonitrile-butadiene), poly(ethylene-vinyl acetate), polyacrylate, and polyethylene terephthalate. In one embodiment, said polymer is selected from the group consisting of poly(acrylonitrile-butadiene), poly(ethylene-vinyl acetate), polyacrylate, and polyester. In one embodiment, said polymer is selected from the group consisting of poly(acrylonitrile-butadiene), poly(ethylene-vinyl acetate), polyethylene terephthalate, and polyester. In one embodiment, said polymer is selected from the group consisting of poly(acrylonitrile-butadiene), polyurethane, synthetic polyamide, and polyacrylate. In one embodiment, said polymer is selected from the group consisting of poly(acrylonitrile-butadiene), polyurethane, synthetic polyamide, and polyethylene terephthalate. In one embodiment, said polymer is selected from the group consisting of poly(acrylonitrile-butadiene), polyurethane, synthetic polyamide, and polyester. In one embodiment, said polymer is selected from the group consisting of poly(acrylonitrile-butadiene), polyurethane, polyacrylate, and polyethylene terephthalate. In one embodiment, said polymer is selected from the group consisting of poly(acrylonitrile-butadiene), polyurethane, polyacrylate, and polyester. In one embodiment, said polymer is selected from the group consisting of poly(acrylonitrile-butadiene), polyurethane, polyethylene terephthalate, and polyester. In one embodiment, said polymer is selected from the group consisting of poly(acrylonitrile-butadiene), synthetic polyamide, polyacrylate, and polyethylene terephthalate. In one embodiment, said polymer is selected from the group consisting of poly(acrylonitrile-butadiene), synthetic polyamide, polyacrylate, and polyester. In one embodiment, said polymer is selected from the group consisting of poly(acrylonitrile-butadiene), synthetic polyamide, polyethylene terephthalate, and polyester. In one embodiment, said polymer is selected from the group consisting of poly(acrylonitrile-butadiene), polyacrylate, polyethylene terephthalate, and polyester. In one embodiment, said polymer is selected from the group consisting of poly(acrylonitrile-butadiene), poly(ethylene-vinyl acetate), polyurethane, synthetic polyamide, and polyacrylate. In one embodiment, said polymer is selected from the group consisting of poly(acrylonitrile-butadiene), poly(ethylene-vinyl acetate), polyurethane, synthetic polyamide, and polyethylene terephthalate. In one embodiment, said polymer is selected from the group consisting of poly(acrylonitrile-butadiene), poly(ethylene-vinyl acetate), polyurethane, synthetic polyamide, and polyester. In one embodiment, said polymer is selected from the group consisting of poly(acrylonitrile-butadiene), poly(ethylene-vinyl acetate), polyurethane, polyacrylate, and polyethylene terephthalate. In one embodiment, said polymer is selected from the group consisting of poly(acrylonitrile-butadiene), poly(ethylene-vinyl acetate), polyurethane, polyacrylate, and polyester. In one embodiment, said polymer is selected from the group consisting of poly(acrylonitrile-butadiene), poly(ethylene-vinyl acetate), polyurethane, polyethylene terephthalate, and polyester. In one embodiment, said polymer is selected from the group consisting of poly(acrylonitrile-butadiene), poly(ethylene-vinyl acetate), synthetic polyamide, polyacrylate, and polyethylene terephthalate. In one embodiment, said polymer is selected from the group consisting of poly(acrylonitrile-butadiene), poly(ethylene-vinyl acetate), synthetic polyamide, polyacrylate, and polyester. In one embodiment, said polymer is selected from the group consisting of poly(acrylonitrile-butadiene), poly(ethylene-vinyl acetate), synthetic polyamide, polyethylene terephthalate, and polyester. In one embodiment, said polymer is selected from the group consisting of poly(acrylonitrile-butadiene), poly(ethylene-vinyl acetate), polyacrylate, polyethylene terephthalate, and polyester. In one embodiment, said polymer is selected from the group consisting of poly(acrylonitrile-butadiene), polyurethane, synthetic polyamide, polyacrylate, and polyethylene terephthalate. In one embodiment, said polymer is selected from the group consisting of poly(acrylonitrile-butadiene), polyurethane, synthetic polyamide, polyacrylate, and polyester. In one embodiment, said polymer is selected from the group consisting of poly(acrylonitrile-butadiene), polyurethane, synthetic polyamide, polyethylene terephthalate, and polyester. In one embodiment, said polymer is selected from the group consisting of poly(acrylonitrile-butadiene), polyurethane, polyacrylate, polyethylene terephthalate, and polyester. In one embodiment, said polymer is selected from the group consisting of poly(acrylonitrile-butadiene), synthetic polyamide, polyacrylate, polyethylene terephthalate, and polyester. In one embodiment, said polymer is selected from the group consisting of poly(acrylonitrile-butadiene), poly(ethylene-vinyl acetate), polyurethane, synthetic polyamide, polyacrylate, and polyethylene terephthalate. In one embodiment, said polymer is selected from the group consisting of poly(acrylonitrile-butadiene), poly(ethylene-vinyl acetate), polyurethane, synthetic polyamide, polyacrylate, and polyester. In one embodiment, said polymer is selected from the group consisting of poly(acrylonitrile-butadiene), poly(ethylene-vinyl acetate), polyurethane, synthetic polyamide, polyethylene terephthalate, and polyester. In one embodiment, said polymer is selected from the group consisting of poly(acrylonitrile-butadiene), poly(ethylene-vinyl acetate), polyurethane, polyacrylate, polyethylene terephthalate, and polyester. In one embodiment, said polymer is selected from the group consisting of poly(acrylonitrile-butadiene), poly(ethylene-vinyl acetate), synthetic polyamide, polyacrylate, polyethylene terephthalate, and polyester. In one embodiment, said polymer is selected from the group consisting of poly(acrylonitrile-butadiene), polyurethane, synthetic polyamide, polyacrylate, polyethylene terephthalate, and polyester. In one embodiment, said polymer is selected from the group consisting of poly(acrylonitrile-butadiene), poly(ethylene-vinyl acetate), polyurethane, synthetic polyamide, polyacrylate, polyethylene terephthalate, and polyester.

As used herein, the term “plastic” is intended to comprise both thermoset and thermoplastic materials. As apparent to a person of skill in the art, synthetic elastomers are comprised in the definition.

While the invention has been described with reference to various exemplary aspects and embodiments, it will be understood by those skilled in the art that various changes may be made and equivalents may be substituted for elements thereof without departing from the scope of the invention. Therefore, it is intended that the invention not be limited to any particular embodiment contemplated, but that the invention will include all embodiments falling within the scope of the appended claims. The invention will be further illustrated by the following non-limiting Examples.

### BRIEF DESCRIPTION OF THE FIGURES

FIG. 1 shows the difference between the mass evolution of larvae in percentage, obtained on PP (cross) and PU (circles) compared to the mass evolution in percentage, obtained on wax. The conditions are described in Example 1.

FIG. 2 shows the difference between the mass evolution of larvae, in percentage obtained on a specific feeding substrate with the difference of mass evolution, in percentage, obtained on wax. The conditions and the specific polymers are given in Example 2.

FIG. 3 shows the difference in percentage between the mass evolution of larvae obtained on a specific feeding substrate in comparison to that of obtained on wax. The conditions and the specific polymers are given in Example 3.

### REFERENCES

Bombelli et al., 2017, Curr. Biol., 27, 8, R292-R293
Lou et al., 2020, Environ. Sci. Technol. 54, 5, 2821-2831

### EXAMPLES

### Example 1

In this example the larvae of G. mellonella were grown on polypropylene (PP), polyurethane (PUR) and wax and the effect of different feeding substrates on larval mass evolution was examined, knowing that wax is the natural feeding substrate for those insects.

The calculations performed were carried out as follows:

$$x = \frac{( m ( 1 ) - m 0 ( 1 ) ) susbtrate}{m 0 ( 1 ) substrate} \times 100 - \frac{( m ( 1 ) - m 0 ( 1 ) ) wax}{m 0 ( l ) wax} \times 100$$

where m(l) stands for average mass of one larva at time t, m0(l) stands for the average initial mass of larva for each respective experiment. X is given in %.

### Material and Methods

The following materials were used as feed (also referred to as substrate in the calculations above):
PP used was from a chocolate packaging, and
PUR was from a kitchen dishwashing sponge.

Paraffin-based wax was from a protection for Bel's group cheese.

The larvae were purchased from Vivara (Sweden), at the 6-7th instar, with an average mass of 380 mg.

To avoid any interference the received larvae were starved for two days before being subjected to the different feed in order to be able to properly appreciate the effect of the newly feed substrate.

10 larvae were placed in round-bottom glass vessels to reach the density of 0.63 larvae per square cm. The temperature was maintained at 24.1+/−0.4° C. and the relative humidity was maintained at 78+/−4%.

All the experiments were performed in triplicates and the results are presented as mean values of the three replicative experiments.

### Obtained Results and Conclusion

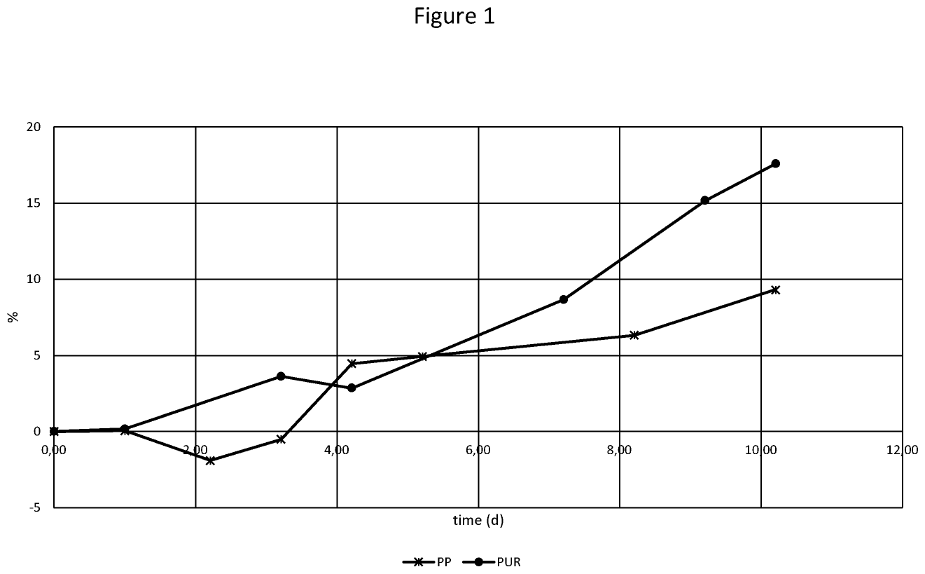

The experiment lasted for 10 days. During the first 4 days there was an adaptation period of larvae towards the new substrates. The mass evolution was lower with PP substrate comparing to wax during this period (FIG. 1). However, after this adaptation period, the mass of the larvae fed the synthetic substrates increased compared to mass of larvae fed wax, the difference being of 9.3% for PP and 17.6% for PUR at the end of the experiment, knowing that wax is the natural feeding substrates for those insects.

Thus, this experiment establishes that PP and PUR are valuable feeding substrates for studied insects and that feeding the larvae said polymers increases growth and mass of the larvae. Hence, it is shown that the larvae feeding on said polymers as the only food source have a desirable mass increase and are able to digest the polymers and thus break down plastic material (such as plastic waste).

### Example 2

In this example the larvae of G. mellonella were grown on different polymers, their mixes, and/or copolymers, and on wax and the effect of different feeding substrates on larval mass evolution examined. The purpose of the experiment was to demonstrate that G. mellonella is able to digest a large variety of plastic polymer materials. The calculations were carried out as outlined in Example 1.

### Material and Methods

The following materials were used as feed (also referred to as substrate in the calculations above):

### Pure Polymers (100%):

Polyamide (PAm) used was from a sweater manufactured by IPEM (Marseille)
Polyester (PEster) was from a pyjama manufactured by Lindex
Polyacrylate (PAcryl) was coming from a hat manufactured by H&M
Cotton was from a sweater manufactured by Benetton.

### Mixes and Copolymers:

PAm/polyether-polyurethane copolymer: 82:18 was from a bath cloth manufactured by Medalist
Cotton/polyester/polyether-polyurethane copolymer: 57:38:5 was from an undercloth manufactured by Joliness.

To avoid any interference the received larvae were starved for two days before the beginning of the differentiate feeding.

20 larvae were placed in round-bottom glass vessels to reach the density of 1.23 larvae per square cm. The temperature was maintained at 23.9+/−0.6° C., and the relative humidity was maintained at 81.4+/−5.1%. All the experiments were performed in duplicates and the results are presented as mean values of the two replicative experiments.

### Obtained Results and Conclusion

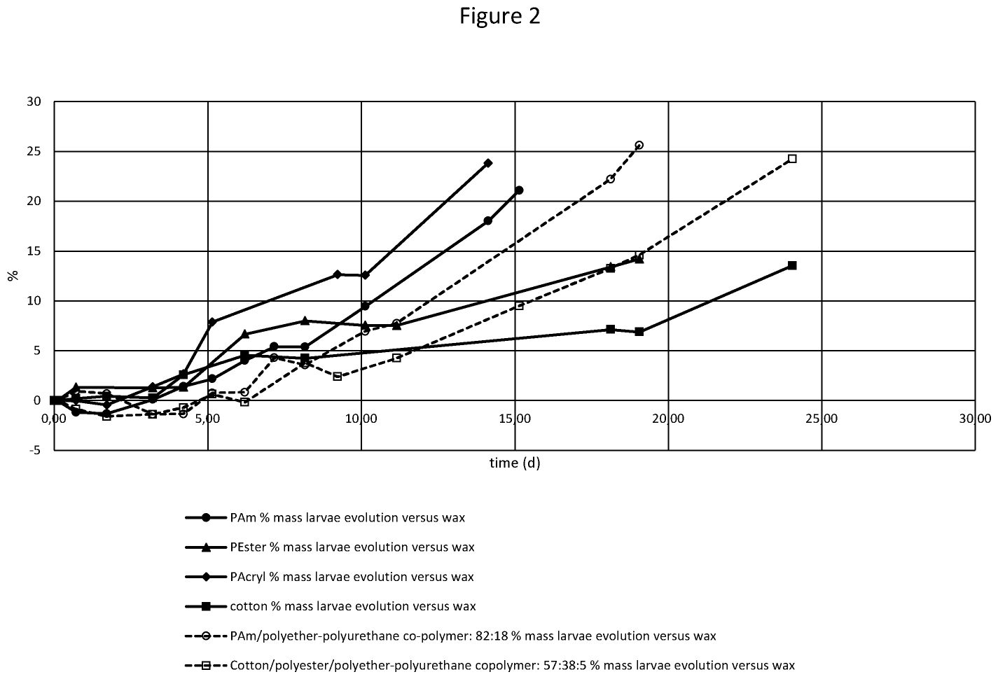

Depending on the substrate the experiment lasted for 14 to 24 days. The individual time point for the end of the experiment is due that that most larvae died of pupated at this point. The obtained results show that there is a need for an adaptation period of 5-6 days for some substrates, e.g., Cotton/polyester/polyether-polyurethane copolymer: 57:38:5 whereas other substrates are directly surperforming comparing to wax, e.g., PAcryl (FIG. 2). This indicates that the larvae require a period to adapt to a subset of plastic polymer based feeds, while other plastic polymer feeds are immediately efficiently utilized as an energy source for the larvae.

By the end of the experiment all the tested substrates show significant surperformance compared to wax. The mass of larvae fed on the tested plastic polymer feed substrates is significantly increased compared to the mass of larvae fed on the wax-based feed, which is the natural feeding substrate for these insect larvae. The data showing the increase in larval mass is summarized in Table 1 below:

TABLE 1
Summary of results at end of experiment. The table summarizes the %
increase in larval mass of the populations of larvae fed on the material
as indicated in the table compared to the control population fed on wax.

| | End of experiment | % increase in |
|---|---|---|
| Substrate (feed) | (day) | mass |
| Polyamide (PAm) | 15 | 21% |
| Polyester (PEster) | 19 | 13% |
| Polyacrylate (PAcryl). | 14 | 24% |
| Cotton | 24 | 13.5% |
| PAm/polyether-polyurethane | 19 | 25% |
| copolymer: 82:18 | | |
| Cotton/polyester/polyether- | 24 | 24% |
| polyurethane copolymer: | | |
| 57:38:5 | | |

Thus, the data indicate that the biomass (as measured by the mass of larvae) increases in the range of from 13% (as shown for PEster) to more than 25% (as shown for PAm/polyether-polyurethane copolymer: 82:18), compared to wax. This is considered a significant increase in the biomass production of the system.

In summary, this experiment establishes that different polymers, their mixes, and copolymers are valuable feeding substrates for studied insects and lead to an increased larval mass compared to natural feed of said insects. It is envisioned that the increase in larval mass may be useful for production of biomass. It was thus demonstrated that G. mellonella is able to digest a large variety of plastic polymer materials.

### Example 3

In this example the larvae of G. mellonella were grown on different polymers, their mixes and copolymers, and on wax. The effect of different feeding substrates on larval mass evolution was examined, knowing that wax is the natural feeding substrates for said insects. The calculations were carried out as outlined in Example 1.

### Material and Methods

The following materials were used as feed (also referred to as substrate in the calculations above):

### Pure Polymers (100%):

PVC (polyvinyl chloride) was from single use gloves manufactured by Bluewear.

### Mixes and Copolymers:

poly(acrylonitrile-butadiene), hereafter called “nitrile”, was from single use gloves PFNB®243 manufactured by Comfort Rubber Gloves Industries SDN;
poly(ethylene-vinyl acetate), hereafter called “EVA”, was from kneepads manufactured by Bluewear;
PVC/PEster, 70%/30%, was from gloves manufactured by Bluewear.
   Paraffin-based wax was from a protection for Bel's group cheese.

The larvae were purchased from Herpers Choise (Sweden), at the 6-7th instar, with an average mass of 320 mg. To avoid any interference the received larvae were starved for two days before the beginning of the differentiate feeding.

20 larvae were placed in square-bottom glass vessels to reach the density of 0.16 larvae per square cm. The temperature was maintained at 23.2+/−0.4 degrees C.

All the experiments were performed in duplicates and the results are presented as mean values of the two replicative experiments.

### Obtained Results and Conclusion

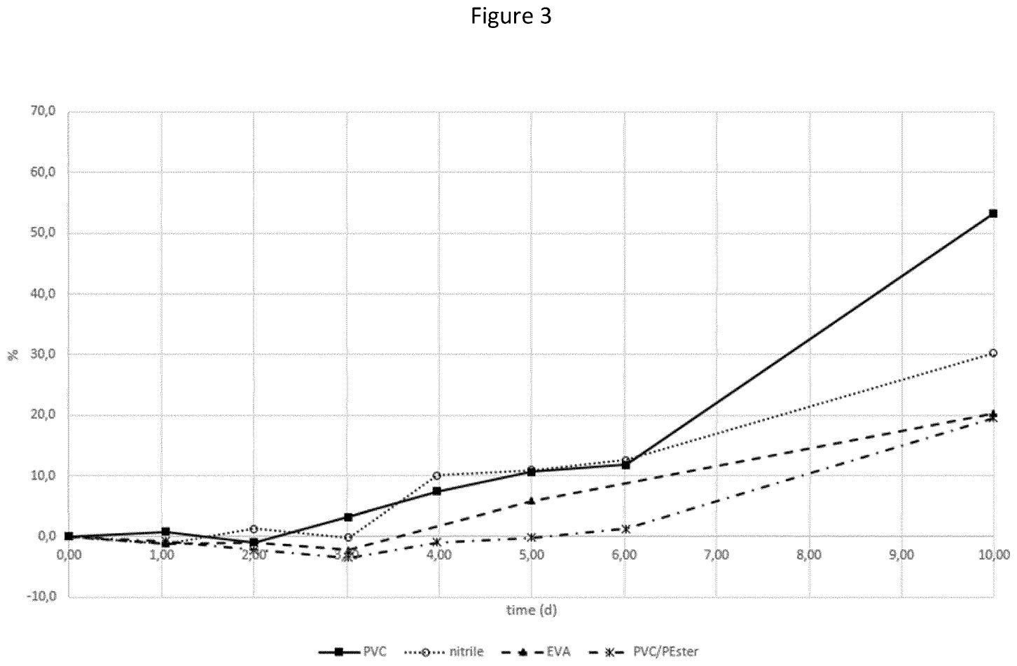

The experiment lasted for 10 days. Two development phases are observed (FIG. 3):

The first phase lasted for 2-5 days and corresponds to an adaptation period for the insects, wherein no significant difference between the mass evolution of insects fed with wax and insects fed with different polymeric materials is observed.

In the second phase, when the adaptation is finished, the mass increase of insects fed with polymeric materials is significantly higher in comparison to that of insects fed with wax. After 10 days of experimentation, the mass increase was about 20% for EVA and PVC/PEster and about 53% for PVC. Thus, the mass increase was in the range of 20-53% for said polymers after 10 days.

Thus, it was surprisingly found that the utilization of different types of polymers, their mixes and copolymers, such as PVC, nitrile, EVA and PVC/Pester, are valuable feeding substrates for the studied insects that lead to an increased larval mass compared to natural feed of said insects. It is concluded that valuable feeding substrates for the studied insects comprise copolymers of polyacrylonitrile and copolymers of polyethylene. It is envisioned that the increase in larval mass may be useful for production of biomass. It was thus demonstrated that G. mellonella is able to digest a large variety of plastic polymer materials such as pure polymers, polymer mixes and copolymers.

### Example 4

In this example the larvae of T. molitor are grown on different polymers, their mixes, and copolymers, and on wheat bran. The effect of different feeding substrates on larval mass evolution is examined, knowing that wheat bran is a natural feeding substrate for those insects.

### Material and Methods

### Pure Polymers (100%):

Polypropylene (PP) is from a three-layer surgical face mask (all three layers were used, including non-woven and melt-blown layers), type IIR, manufactured by ESound Med;
Polyamide (PAm) is from a sweater manufactured by IPEM (Marseille),
Polyester (PEster) is from a pyjama manufactured by Lindex,
Polyacrylate (PAcryl) is coming from a hat manufactured by H&M,
Cotton is from a sweater manufactured by Benetton;
Polyethylene (PE) is from a dipers' packaging manufactured by Libero

### Mixes and Copolymers:

PEster/PAm/polyether-polyurethane copolymer: 80:10:10 is from an undercloth manufactured by Man Underwear;
Cotton/polyester/polyether-polyurethane copolymer: 57:38:5 is from an undercloth manufactured by Joliness

To avoid any interference the received larvae are starved for two days before the beginning of the differentiate feeding.

20 larvae are placed in round-bottom glass vessels to reach the density of 1+/−0.5 larvae per square cm. The temperature is maintained at 27+/−2° C., and the relative humidity is maintained at 80+/−5%.

All the experiments are performed in duplicates and the results are presented as mean values of the two replicative experiments.

### Expected Results and Conclusion

It is expected that larvae need an adaptation period of several days to adjust to the new diets, after this adaptation period the larvae are expected to perform at least as well on the synthetic substrates as on wheat bran.

Thus, this experiment is expected to establish that the polymers, their mixes, and copolymers are valuable feeding substrates for those insects.

### Example 5

In this example the larvae of A. diaperinus are grown on different polymers, their mixes, and copolymers, and on wheat bran. The effect of different feeding substrates on larval mass evolution is examined, knowing that wheat bran is a natural feeding substrate for those insects.

### Material and Methods

### Pure Polymers (100%):

Polypropylene (PP) is from a three-layer surgical face mask (all three layers were used, including non-woven and melt-blown layers), type IIR, manufactured by ESound Med;
Polyamide (PAm) is from a sweater manufactured by IPEM (Marseille),
Polyester (PEster) is from a pyjama manufactured by Lindex,
Polyacrylate (PAcryl) is coming from a hat manufactured by H&M,
Cotton is from a sweater manufactured by Benetton;
Polyethylene (PE) is from a dipers' packaging manufactured by Libero

### Mixes and Copolymers:

PEster/PAm/polyether-polyurethane copolymer: 80:10:10 is from an undercloth manufactured by Man Underwear;
Cotton/polyester/polyether-polyurethane copolymer: 57:38:5 is from an undercloth manufactured by Joliness

To avoid any interference the received larvae are starved for two days before the beginning of the differentiate feeding.

20 larvae are placed in round-bottom glass vessels to reach the density of 1+/−0.5 larvae per square cm. The temperature is maintained at 27+/−2° C., and the relative humidity is maintained at 80+/−5%.

All the experiments are performed in duplicates and the results are presented as mean values of the two replicative experiments.

### Expected Results and Conclusion

It is expected that larvae need an adaptation period of several days to adjust to the new diets, after this adaptation period the larvae are expected to perform at least as well on the synthetic substrates as on wheat bran.

Thus, this experiment is expected to establish that the polymers, their mixes, and copolymers are valuable feeding substrates for said insects.

### ITEMIZED LIST OF EMBODIMENTS

1. Method for degrading one or more plastic-comprising materials, the method comprising
contacting at least one larva of the species selected from the group consisting of Galleria mellonella, Tenebrio molitor and Alphitobius diaperinus with at least one or more plastic—comprising materials under conditions permissive for said at least one larva to digest said material, wherein said material comprises at least one plastic polymer selected from the group consisting of polyethylene terephthalate, polypropylene, polypropylene terephthalate, polyether, polyurethane, polyester, polyacrylate, polyglycol, polyvinyl chloride, polycarbonate, polyvinylidene chloride, synthetic polyamide and any mixes or co-polymers thereof.

2. Method for degrading one or more plastic-comprising materials, the method comprising
contacting at least one larva of the species selected from the group consisting of Galleria mellonella, Tenebrio molitor and Alphitobius diaperinus with at least one or more plastic-comprising materials under conditions permissive for said at least one larva to digest said material, wherein said material comprises at least one plastic polymer selected from the group consisting of polyethylene terephthalate, polypropylene terephthalate, polyether, polyurethane, polyester, polyacrylate, polyglycol, polyvinyl chloride, polyvinyl acetate, polycarbonate, polyvinylidene chloride, polyacrylonitrile, synthetic polyamide and any mixes or copolymers thereof, and any copolymers of polyethylene,
such as wherein said material comprises at least one plastic polymer selected from the group consisting of polyethylene terephthalate, polypropylene terephthalate, polyether, polyurethane, polyester, polyacrylate, polyglycol, polyvinyl chloride, polycarbonate, polyvinylidene chloride, polyacrylonitrile, synthetic polyamide and any mixes or copolymers thereof, and any copolymers of polyethylene.

3. Method according to any one of the preceding items, wherein said plastic-comprising material consists of at least one plastic polymer selected from the group consisting of polyethylene terephthalate, polypropylene terephthalate, polyether, polyurethane, polyester, polyacrylate, polyglycol, polyvinyl chloride, polyvinyl acetate, polycarbonate, polyvinylidene chloride, polyacrylonitrile, synthetic polyamide and any mixes or copolymers thereof, and any copolymers of polyethylene,
such as the group consisting of polyethylene terephthalate, polypropylene terephthalate, polyether, polyurethane, polyester, polyacrylate, polyglycol, polyvinyl chloride, polycarbonate, polyvinylidene chloride, synthetic polyamide and any mixes or copolymers thereof.

4. Method according to any one of the preceding items, wherein biomass is obtained from said digestion.

5. Method according to item 4, wherein said biomass comprises pupae, and/or larvae of the species selected from the group consisting of Galleria mellonella, Tenebrio molitor and Alphitobius diaperinus, such as the group of Galleria mellonella, and Alphitobius diaperinus, such as the group of Galleria mellonella, and/or feces of said larvae.

6. Method according to any one of items 2 to 5, wherein said polymer is selected from the group consisting of polyurethane, synthetic polyamide, polyester, polyacrylate, polyvinyl chloride, polyacrylonitrile, and any mixes or copolymers thereof, and any copolymers of polyethylene, such as the group consisting of polyurethane, synthetic polyamide, polyester, polyacrylate, and any mixes or copolymers thereof.

7. Method according to any one items 2 to 6, wherein said polymer is selected from the group consisting of polyurethane, synthetic polyamide, polyester, polyvinyl chloride, polyacrylonitrile, and any mixes or copolymers thereof, and any copolymers of polyethylene,
such as the group consisting of polyurethane, synthetic polyamide, polyester, and any mixes or copolymers thereof.

8. Method according to any one of items 2 to 6, wherein said polymer is selected from the group consisting of polyurethane, polyvinyl chloride, polyacrylonitrile, polyacrylate, and any mixes or copolymers thereof, and any copolymers of polyethylene,
such as the group consisting of polyurethane, polyacrylate, and any mixes or copolymers thereof.

9. Method according to any one of items 2 to 8, wherein said polymer is selected from the group consisting of polyurethane, polyvinyl chloride, polyacrylonitrile, and any mixes or copolymers thereof, and any copolymers of polyethylene,
such as the group consisting of polyurethane, and any mixes or copolymers thereof, such as the group consisting of polyurethane.

10. Method according to any one of items 2 to 6, wherein said polymer is selected from the group consisting of synthetic polyamide, polyester, polyacrylate, polyvinyl chloride, polyacrylonitrile, and any mixes or copolymers thereof, and any copolymers of polyethylene,
such as the group consisting of synthetic polyamide, polyester, and polyacrylate, and any mixes or copolymers thereof, such as the group consisting of synthetic polyamide, polyester, and polyacrylate.

11. Method according to any one of the preceding items, wherein said material comprises a further polymer.

12. Method according to any one of items 2 to 11, wherein said copolymer of polyethylene is poly(ethylene-vinyl acetate).

13. Method according to any one of items 1 to 3, wherein said polymer consists of a copolymer comprising polyether and polyurethane, wherein said material optionally comprises a further polymer selected from the list consisting of a polyamide, cellulose, and polyester.

14. Method according to any one of the preceding items, wherein said polymer is a synthetic polymer.

15. Method according to any one of items 1 to 8 and 10 to 14, wherein said polyamide is selected from the group consisting of PA 6; PA 6.6; PA 10; and PA 12.

16. Method according to any one of items 1 to 7 and 10 to 15, wherein said polyester is selected from the group consisting of polyethylene terephthalate, polypropylene terephthalate, polycaprolactone, and polyethylene adipate.

17. Method according to any one of items 1 to 11, wherein said polymer is a polyacrylonitrile copolymer, such as poly(acrylonitrile-butadiene).

18. Method according to any one of items 1 to 11, wherein said polymer is polyvinyl chloride, and wherein said material optionally comprises a further polymer, such as polyester.

19. Method according any one of the preceding items, wherein said method is performed at a temperature of approximately from 4° C. to 40° C., such as approximately from 15° C. to 35° C., preferably of approximately from 20° C. to 30° C., most preferably of approximately from 25° C. to 27° C.

20. Method according any one of the preceding items, wherein said method is performed at a humidity of approximately of from 30% to 99%.

21. Method according to item 20, wherein said humidity is maintained at approximately 80.0%+/−5%.

22. Method according to any one of the preceding items, wherein the larva are kept at a density of 0.2-5 larvae per cm², such as 0.2-3 larvae per cm², such as 0.2-2 larvae per cm² or such as 0.5-3 larvae per cm², such as 0.5-2 larvae per cm², such as 1-2 larvae per cm.²

23. Method according any one of the preceding items, wherein said larva is subjected to starvation prior to contacting the larva with any feed.

24. Method according to item 23, wherein said starvation is for approximately 1 to 20 days, such as approximately for 1 to 10, such as approximately for 1 to 5 days, such as approximately for 2 days.

25. Method according to any one of the preceding items, wherein said larva is of the species selected from the group consisting of Galleria mellonella, and Alphitobius diaperinus.

26. Method according to any one of the preceding items, wherein said larva is of the species G. mellonella or Alphitobius diaperinus.

27. Method according to any one of the preceding items, wherein said larva is of the species G. mellonella.

28. Use of at least one larva of the species selected from the group consisting of Galleria mellonella, Tenebrio molitor and Alphitobius diaperinus, for digestion of a plastic-comprising material, wherein said material comprises or consists of at least one plastic polymer selected from the group consisting of polyethylene terephthalate, polypropylene, polypropylene terephthalate, polyether, polyurethane, polyester, polyacrylate, polyglycol, polyvinyl chloride, polycarbonate, polyvinylidene chloride, synthetic polyamide and any mixes or co-polymers thereof.

29. Use of at least one larva of the species selected from the group consisting of Galleria mellonella, Tenebrio molitor and Alphitobius diaperinus, for digestion of a plastic-comprising material, wherein said material comprises or consists of at least one plastic polymer selected from the group consisting of
polyethylene terephthalate, polypropylene terephthalate, polyether, polyurethane, polyester, polyacrylate, polyglycol, polyvinyl chloride, polyvinyl acetate, polycarbonate, polyvinylidene chloride, polyacrylonitrile, synthetic polyamide and any mixes or copolymers thereof, and any copolymers of polyethylene,
such as wherein said material comprises or consists of at least one plastic polymer selected from the group consisting of polyethylene terephthalate, polypropylene terephthalate, polyether, polyurethane, polyester, polyacrylate, polyglycol, polyvinyl chloride, polycarbonate, polyvinylidene chloride, polyacrylonitrile, synthetic polyamide and any mixes or copolymers thereof, and any copolymers of polyethylene.

30. Use according to item 29, wherein said polymer is selected from the group consisting of polyurethane, synthetic polyamide, polyester, polyacrylate, polyvinyl chloride, polyacrylonitrile, and any mixes or copolymers thereof, and any copolymers of polyethylene,
such as the group consisting of polyurethane, synthetic polyamide, polyester, polyacrylate, and any mixes or copolymers thereof.

31. Use according to any one of items 29 to 30, wherein said polymer is selected from the group consisting of polyurethane, synthetic polyamide, polyester, polyvinyl chloride, polyacrylonitrile, and any mixes or copolymers thereof, and any copolymers of polyethylene,
such as the group consisting of polyurethane, synthetic polyamide, polyester, and any mixes or copolymers thereof.

32. Use according to any one of items 29 to 30, wherein said polymer is selected from the group consisting of polyurethane, polyvinyl chloride, polyacrylonitrile, and polyacrylate, and any mixes or copolymers thereof, and any copolymers of polyethylene,
such as the group consisting of polyurethane, polyacrylate, and any mixes or copolymers thereof.

33. Use according any one of items 29 to 32, wherein said polymer is selected from the group consisting of polyurethane, polyvinyl chloride, polyacrylonitrile, and any mixes or copolymers thereof, and any copolymers of polyethylene,
such as the group consisting of polyurethane, and any mixes or copolymers thereof, such as wherein the polymer is polyurethane.

34. Use according to any one of items 29 to 30 wherein said polymer is selected from the group consisting of synthetic polyamide, polyester, polyacrylate, polyvinyl chloride, polyacrylonitrile, and any mixes or copolymers thereof, and any copolymers of polyethylene,
such as the group consisting of synthetic polyamide, polyester, and polyacrylate, and any mixes or copolymers thereof, such as the group consisting of synthetic polyamide, polyester, and polyacrylate.

35. Use according to item 33, wherein said polymer is a copolymer comprising polyether and polyurethane, optionally comprising a further polymer selected from the list consisting of a synthetic polyamide, cellulose and polyester.

36. Use according to any one of items 28 to 31 and 33 to 34, wherein said polyester is selected from the group consisting of polyethylene terephthalate, polypropylene terephthalate, polycaprolactone, and polyethylene adipate.

37. Use according to any one of items 28 to 31 and 34 to 35, wherein said polyamide is selected from the group consisting of PA 6; PA 6.6; PA 10; and PA 12.

38. Product obtained by the method according to any one of items 1 to 27, wherein said product comprises biomass.

39. Product according to item 38, wherein said product consist of biomass.

40. Product according to any one of items 38 to 39, wherein said biomass comprises pupae, and/or larvae of the species selected from the group consisting of Galleria mellonella, Tenebrio molitor and Alphitobius diaperinus, such as selected from the group consisting of Galleria mellonella, and Alphitobius diaperinus, such as selected from the group consisting of Galleria mellonella, and/or feces of said larvae.

41. Product according to any one of items 38 to 40, which product is a fertilizer.

42. Product according to any one of items 38 to 40, which product is an animal feed.

1. Method for degrading one or more plastic-comprising material, the method comprising
contacting at least one larva of the species selected from the group consisting of Galleria mellonella and Alphitobius diaperinus, with at least one or more plastic-comprising materials under conditions permissive for said at least one larva to digest said material, wherein said material comprises at least one plastic polymer selected from the group consisting of polyethylene terephthalate, polypropylene terephthalate, polyether, polyurethane, polyester, polyacrylate, polyglycol, polyvinyl chloride, polycarbonate, polyvinylidene chloride, polyacrylonitrile, synthetic polyamide and any mixes or copolymers thereof, and any copolymers of polyethylene;
such as the group consisting of polyethylene terephthalate, polypropylene terephthalate, polyether, polyurethane, polyester, polyacrylate, polyglycol, polyvinyl chloride, polycarbonate, polyvinylidene chloride, synthetic polyamide and any mixes or copolymers thereof.

2. Method according to claim 1, wherein said plastic-comprising material consists of at least one plastic polymer selected from the group consisting of polyethylene terephthalate, polypropylene terephthalate, polyether, polyurethane, polyester, polyacrylate, polyglycol, polyvinyl chloride, polycarbonate, polyvinylidene chloride, polyacrylonitrile, synthetic polyamide and any mixes or copolymers thereof, and any copolymers of polyethylene;
such as the group consisting of polyethylene terephthalate, polypropylene terephthalate, polyether, polyurethane, polyester, polyacrylate, polyglycol, polyvinyl chloride, polycarbonate, polyvinylidene chloride, synthetic polyamide and any mixes or copolymers thereof.

3. Method according to claim 1, wherein said polymer is selected from the group consisting of polyurethane, synthetic polyamide, polyester, polyacrylate, polyvinyl chloride, polyacrylonitrile, and any mixes or copolymers thereof, and any copolymers of polyethylene;
such as the group consisting of polyurethane, synthetic polyamide, polyester, polyacrylate, and any mixes or copolymers thereof.

4. Method according to claim 1, wherein said polymer is selected from the group consisting of polyurethane, polyvinyl chloride, polyacrylonitrile, polyacrylate, and any mixes or copolymers thereof, and any copolymers of polyethylene;
such as the group consisting of polyurethane, polyacrylate, and any mixes or copolymers thereof.

5. Method according to claim 1, wherein
said polymer is selected from the group consisting of synthetic polyamide, polyester, polyacrylate, polyvinyl chloride, polyacrylonitrile, and any mixes or copolymers thereof, and any copolymers of polyethylene;
such as the group consisting of synthetic polyamide, polyester, and polyacrylate, and any mixes or copolymers thereof;
such as the group consisting of synthetic polyamide and polyacrylate.

6. Method according to claim 1, wherein said material comprises a further polymer.

7. Method according to claim 1, wherein
said polymer is a copolymer comprising polyether and polyurethane, optionally wherein said material comprises a further polymer selected from the list consisting of a polyamide, cellulose, and polyester.

8. Method according to claim 1, wherein said synthetic polyamide is selected from the group consisting of PA 6; PA 6.6; PA 10; and PA 12.

9. Method according to claim 1, wherein said polyester is selected from the group consisting of polyethylene terephthalate, polypropylene terephthalate, polycaprolactone, and polyethylene adipate.

10. Method according to claim 1, wherein said at least one larva is of the species G. mellonella.

11. Method according to claim 1, wherein biomass is obtained from said digestion.

12. Use of at least one larva of the species selected from the group consisting of Galleria mellonella and Alphitobius diaperinus for digestion of a plastic-comprising material, wherein said material comprises at least one plastic polymer selected from the group consisting of polyethylene terephthalate, polypropylene terephthalate, polyether, polyurethane, polyester, polyacrylate, polyglycol, polyvinyl chloride, polycarbonate, polyvinylidene chloride, polyacrylonitrile, synthetic polyamide, and any mixes or copolymers thereof, and any copolymers of polyethylene;
such as polyethylene terephthalate, polypropylene terephthalate, polyether, polyurethane, polyester, polyacrylate, polyglycol, polyvinyl chloride, polycarbonate, polyvinylidene chloride, synthetic polyamide and any mixes or copolymers thereof.

13. Use according to claim 12, wherein said polymer is selected from the group consisting of polyurethane, synthetic polyamide, polyester, polyacrylate, polyvinyl chloride, polyacrylonitrile, and any mixes or copolymers thereof, and any copolymers of polyethylene;
such as is selected from the group consisting of polyurethane, synthetic polyamide, polyester, polyacrylate, and any mixes or copolymers thereof.

14. Use according to claim 12, wherein said at least one larva is of the species G. mellonella.

15. Product obtained by the method according to claim 1, wherein said product comprises biomass.
